SEQUENCE LISTING (51 sequences)

SEQ ID NO: 1 - protein, 418 aa, Homo sapiens [sequence omitted]

SEQ ID NO: 2 - DNA, 21 bp, Artificial Sequence
  Synthetic construct, RT-PCR primer
accatgagagctgtccctctg

SEQ ID NO: 3 - DNA, 18 bp, Artificial Sequence
  Synthetic construct, RT-PCR primer
tcagatgttccgatacac

SEQ ID NO: 4 - DNA, 15 bp, Artificial Sequence
  Synthetic construct,  primer
atgagagctgcaccc

SEQ ID NO: 5 - DNA, 21 bp, Artificial Sequence
  Synthetic construct
gctctgggcagattcaaaagg

SEQ ID NO: 6 - DNA, 26 bp, Artificial Sequence
  Synthetic construct, primer
ttaggatccaccatgagagctgcacc

SEQ ID NO: 7 - DNA, 27 bp, Artificial Sequence
  Synthetic construct, primer
aatggatccgatgttcccataggattc

SEQ ID NO: 8 - DNA, 19 bp, Homo sapiens
aacagccatcaaggtactg

SEQ ID NO: 9 - DNA, 24 bp, Homo sapiens
tttcccacagttctaaaggtcatc

SEQ ID NO: 10 - protein, 5 aa, Homo sapiens
VLKVI

SEQ ID NO: 11 - DNA, 13 bp, Homo sapiens
aacagccatcaag

SEQ ID NO: 12 - DNA, 23 bp, Homo sapiens
gtactgaagacagtacagattag

SEQ ID NO: 13 - protein, 7 aa, Homo sapiens
GTEDSTD

SEQ ID NO: 14 - DNA, 19 bp, Homo sapiens
agcagccaccaaggtactg

SEQ ID NO: 15 - DNA, 24 bp, Homo sapiens
tcggtcgcagtgctgagggttatc

SEQ ID NO: 16 - protein, 5 aa, Homo sapiens
VRLVI

SEQ ID NO: 17 - DNA, 19 bp, Homo sapiens
agcagccaccaaggtactg

SEQ ID NO: 18 - DNA, 21 bp, Homo sapiens
gcttgagcaggacggaattaa

SEQ ID NO: 19 - DNA, 19 bp, Homo sapiens
agcaaccaccaaggtacag

SEQ ID NO: 20 - DNA, 24 bp, Homo sapiens
cgttccgcagtgctgagggtcatc

SEQ ID NO: 21 - protein, 5 aa, Homo sapiens
VLRVI

SEQ ID NO: 22 - DNA, 19 bp, Homo sapiens
agcaaccaccaaggtacag

SEQ ID NO: 23 - DNA, 21 bp, Homo sapiens
tttgtttcagcgtcgctgtaa

SEQ ID NO: 24 - protein, 61 aa, Homo sapiens
MRAAPLLLARAASLSLGFLFLLFFWLDRSVLAKELKFVTLVFRHGDRSPIDTFPTDPIKES

SEQ ID NO: 25 - protein, 60 aa, Mus sp.
MRAVPLPLSRTASLSLGFLLLLSLCLDPGQAKELKFVTLVFRHGDRGPIETFPTDPITES

SEQ ID NO: 26 - protein, 60 aa, Rattus rattus
MRAVPLHLVGTASLTLGFLLLLSLRLDPGQAKELKFVTLVFRHGDRGPIETFPNDPIKES

SEQ ID NO: 27 - protein, 59 aa, Homo sapiens
MAGKRSGWSRAALLQLLLGVNLVVMPPTRARSLRFVTLLYRHGDRSPVKTYPKDPYQEE

SEQ ID NO: 28 - protein, 59 aa, Mus sp.
MAGRQTGWSQAALLQFLLGMCLTVMPPIQARSLRFVTLLYRHGDRSPVKTYPKDPYQEE

SEQ ID NO: 29 - protein, 59 aa, Rattus rattus
MAGRQSGWSQAALLQFLLGMCLMVMPPIQARSLRFVTLLYRHGDRSPVKAYPKDPYQEE

SEQ ID NO: 30 - protein, 70 aa, Homo sapiens
MRLWTPVGVLTSLAYCLHQRRVALAELQEADGQCPVDRSLLKLKMVQVVFRHGARSPLKPLPLEEQVEWN

SEQ ID NO: 31 - protein, 55 aa, Homo sapiens
IPQDWSTECMTTNSHQVLKVIFAVAFCLISAVLMVLLFIHIRRGLCWQRESYGNI

SEQ ID NO: 32 - protein, 55 aa, Mus sp.
ISQDWATECMATSSHQVLRVILATTFCLVTGILVILLLVLIRHGPCWQRDVYRNI

SEQ ID NO: 33 - protein, 55 aa, Rattus rattus
IPQDWATECMGTSNHQVLRVILATTFCLVTGILVILLLVLIRHGPCWQRDVYRNI

SEQ ID NO: 34 - protein, 62 aa, Homo sapiens
VPKDWQQECQLASGPADTEVIVALAVCGSILFLLIVLLLTVLFRMQAQPPGYRHVADGEDHA

SEQ ID NO: 35 - protein, 62 aa, Mus sp.
IPKDWQKECQLANDTADTEVIVALAVCGSILFLLIVLLLTILFRMQAQPPGYHHVADREDHA

SEQ ID NO: 36 - protein, 62 aa, Rattus rattus
IPKDWQKECQLASDTADTEVIVALAVCGSILFLLIVLLLTVLFRMQAQPPGYHHVADREDHA

SEQ ID NO: 37 - protein, 24 aa, Homo sapiens
YTLSPEKYHALCSQTQVMEVGNEE

SEQ ID NO: 38 - protein, 418 aa, Homo sapiens [sequence omitted]

SEQ ID NO: 39 - protein, 417 aa, Mus sp.
MRAVPLPLSRTASLSLGFLLLLSLCLDPGQAKELKFVTLVFRHGDRGPIETFPTDPITESSWPQGFGQLTQWGMEQHYELGSYIRKRYGRFLNDTYKHDQIYIRSTDVDRTLMSAMTNLAALFPPEGISIWNPRLLWQPIPVHTVSLSEDRLLYLPFRDCPRFEELKSETLESEEFLKRLHPYKSFLDTLSSLSGFDDQDLFGIWSKVYDPLFCESVHNFTLPSWATEDAMIKLKELSELSLLSLYGIHKQKEKSRLQGGVLVNEILKNMKLATQPQKYKKLVMYSAHDTTVSGLQMALDVYNGVLPPYASCHMMELYHDKGGHFVEMYYRNETQNEPYPLTLPGCTHSCPLEKFAELLDPVISQDWATECMATSSHQVLRVILATTFCLVTGILVILLLVLIRHGPCWQRDVYRNI

SEQ ID NO: 40 - protein, 417 aa, Rattus rattus
MRAVPLHLVGTASLTLGFLLLLSLRLDPGQAKELKFVTLVFRHGDRGPIETFPNDPIKESSWPQGFGQLTKWGMGQHYELGSYIRRRYGRFLNNSYKHDQVYIRSTDVDRTLMSAMTNLAALFPPEGISIWNPRLLWQPIPVHTVSLSEDRLLYLPFRDCPRFQELKSETLKSEEFLKRLQPYKSFIDTLPSLSGFEDQDLFEIWSRLYDPLYCESVHNFTFRTWATEDAMTKLKELSELSLLSLYGIHKQKEKSRLQGGVLVNEILKNMKLATQPQKARKLIMYSAYDTTVSGLQMALELYNGLLPPYASCHIMELYQDNGGTFVEMYYRNETQNEPYPLTLPGCTHSCPLEKFAELLDPVIPQDWATECMGTSNHQVLRVILATTFCLVTGILVILLLVLIRHGPCWQRDVYRNI

SEQ ID NO: 41 - protein, 386 aa, Homo sapiens
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 42 - protein, 381 aa, Mus sp.
MRAVPLPLSRTASLSLGFLLLLSLCLDPGQAKELKFVTLVFRHGDRGPIETFPTDPITESSWPQGFGQLTQWGMEQHYELGSYIRKRYGRFLNDTYKHDQIYIRSTDVDRTLMSAMTNLAALFPPEGISIWNPRLLWQPIPVHTVSLSEDRLLYLPFRDCPRFEELKSETLESEEFLKRLHPYKSFLDTLSSLSGFDDQDLFGIWSKVYDPLFCESVHNFTLPSWATEDAMIKLKELSELSLLSLYGIHKQKEKSRLQGGVLVNEILKNMKLATQPQKYKKLVMYSAHDTTVSGLQMALDVYNGVLPPYASCHMMELYHDKGGHFVEMYYRNETQNEPYPLTLPGCTHSCPLEKFAELLDPVISQDWATECMATSSHQGRN

SEQ ID NO: 43 - protein, 381 aa, Rattus rattus
MRAVPLHLVGTASLTLGFLLLLSLRLDPGQAKELKFVTLVFRHGDRGPIETFPNDPIKESSWPQGFGQLTKWGMGQHYELGSYIRRRYGRFLNNSYKHDQVYIRSTDVDRTLMSAMTNLAALFPPEGISIWNPRLLWQPIPVHTVSLSEDRLLYLPFRDCPRFQELKSETLKSEEFLKRLQPYKSFIDTLPSLSGFEDQDLFEIWSRLYDPLYCESVHNFTFRTWATEDAMTKLKELSELSLLSLYGIHKQKEKSRLQGGVLVNEILKNMKLATQPQKARKLIMYSAYDTTVSGLQMALELYNGLLPPYASCHIMELYQDNGGTFVEMYYRNETQNEPYPLTLPGCTHSCPLEKFAELLDPVIPQDWATECMGTSNHQASL

SEQ ID NO: 44 - DNA, 980 bp, Homo sapiens
gggcttcaggacaattcgtgatggcgggggctggttccgccgctgtatcgggggcagggaccccggtggcggggcccacaggccgcgaccttttcgccgaagggctgctggagttcctgcgacccgctgtgcagcagctcgactctcacgtacacgccgtcagagagagccaggtagagctccgggaacaaattgacaacctagccacagaactgtgccgcataaatgaggatcagaaggtggccctggatcttgacccctatgttaagaagctacttaatgcccggcgacgcgttgtcttggttaacaacattctacagaatgctcaggaacgactgagacggctaaaccacagtgttgccaaggaaacagcccgcaggagagcaatgctggattcgggaatttacccccctggctccccaggcaaataacagatgagcctatggactcagtagcacaagtactgttccccagctgccttgtttcaacagacatgcaaagatcctaggagacagtccccatagaccttcagacattaaaaagggagccgtacagtttgtttgaagcacttcgtcttacccatttatgtaggggccccaggaaacctacacacagccagaatgaggttcccaaaggacttacattaattatggctcttgcttcctttcacaaatgagctgaggcctctacttttttttttttaagctgcatacttgaggcttaccttcttcaggactagttaaccagaggggcttcctttgtatgttacatgcctggttacatgggcctggacagcatgtcctctacctgtgacttctcattttcctgtttacactggggatttggagggggcaggcaaagtcaaagtgaatgacctctgtccacccacttttttattgcactggcttgaatacagtagcagtgttgatagaatcattttattcaataaatacttaaaatgaaaaaaaaaaaaaaaaaaa

SEQ ID NO: 45 - protein, 312 aa, Homo sapiens
MAGAGSAAVSGAGTPVAGPTGRDLFAEGLLEFLRPAVQQLDSHVHAVRESQVELREQIDNLATELCRINEDQKVALDLDPYVKKLLNARRRVVLVNNILQNAQERLRRLNHSVAKETARRRAMLDSGIYPPGSPGKQMSLWTQHKYCSPAALFQQTCKDPRRQSPTFRHKGSRTVCLKHFVLPIYVGAPGNLHTARMRFPKDLHLWLLLPFTNELRPLLFFFAAYLRLTFFRTSPEGLPLYVTCLVTWAWTACPLPVTSHFPVYTGDLEGAGKVKVNDLCPPTFLLHWLEYSSSVDRIILFNKYLKKKKKKK

SEQ ID NO: 46 - DNA, 980 bp, Homo sapiens; identical to SEQ ID NO: 44

SEQ ID NO: 47 - protein, 312 aa, Homo sapiens; identical to SEQ ID NO: 45

SEQ ID NO: 48 - DNA, 980 bp, Homo sapiens; identical to SEQ ID NO: 44

SEQ ID NO: 49 - protein, 154 aa, Homo sapiens
MAGAGSAAVSGAGTPVAGPTGRDLFAEGLLEFLRPAVQQLDSHVHAVRESQVELREQIDNLATELCRINEDQKVALDLDPYVKKLLNARRRVVLVNNILQNAQERLRRLNHSVAKETARRRAMLDSGIYPPGSPGKQMSLWTQHKYCSPAALFQ

SEQ ID NO: 50 - DNA, 25 bp, Homo sapiens
aatctagaccatgccagccgttcct

SEQ ID NO: 51 - DNA, 28 bp, Homo sapiens
cacatagccttgttagactagatctctc

(19) United States
(12) Patent Application Publication
(10) Pub. No.: US 2013/0108609 A1
(43) Pub. Date: May 2, 2013

(54) TRANSMEMBRANE PROSTATIC ACID PHOSPHATASE

(71) Applicant: Chempath Oy, Helsinki (FI)

(72) Inventor: Pirkko Vihko, Helsinki (FI)

(73) Assignee: CHEMPATH OY, Helsinki (FI)

(21) Appl. No.: 13/661,616

(22) Filed: Oct. 26, 2012

Related U.S. Application Data
(63) Continuation-in-part of application No. 12/665,232, filed on May 20, 2010, now abandoned.

(30) Foreign Application Priority Data
Jun. 19, 2007 (FI) 20075466

(51) Int. Cl. C12N 9/16

(52) U.S. Cl. CPC C12N 9/16
USPC 424/94.6; 514/44 R

(57) ABSTRACT

The present invention relates to a novel transmembrane prostatic acid phosphatase (TM-PAP) protein or the C-terminal part thereof, nucleic acid molecules encoding said protein, vectors containing said nucleic acid molecules and host cells expressing said proteins. The present invention relates also to pharmaceutical compositions containing TM-PAP or the C-terminal part thereof and methods for using thereof in therapy and diagnostics. The present invention also relates to methods utilizing a transmembrane prostatic acid phosphatase knockout/knockdown non-human animal model and uses thereof.

### CROSS REFERENCE TO RELATED APPLICATIONS

This application is a continuation-in-part of U.S. patent application Ser. No. 12/665,232, which was assigned a 371 date of May 20, 2010 and was a 35 USC §371 national stage entry of PCT/FI08/50377, which was filed Jun. 19, 2008 and claims the benefit of FI 20075466, which was filed Jun. 19, 2007, all of which are incorporated herein by reference as if fully set forth.

### FIELD OF INVENTION

The present invention relates to a novel transmembrane prostatic acid phosphatase (TM-PAP) protein, nucleic acid molecules encoding said protein, vectors containing said nucleic acid molecules and host cells expressing said proteins. The present invention relates also to pharmaceutical compositions containing TM-PAP and methods for using thereof in therapy and diagnostics. The present invention also relates to methods utilizing a transmembrane prostatic acid phosphatase knockout/knockdown non-human animal model.

### BACKGROUND OF THE INVENTION

Prostate cancer is the most common cancer in men and the second leading cause of cancer death in the Western countries. Although chemotherapy treatments have shown survival benefit for hormone-refractory prostate cancer, new and more effective therapies are needed. A novel approach refers to the activation of the immune system using an antigen loaded in antigen-presenting cells (Srivastava P K. Therapeutic cancer vaccines. Curr Opin Immunol 2006; 18: 201-5). Some of the new ongoing vaccine therapies in trial for prostate cancer treatment are based on the essential restriction of PAP (ACPP, EC 3.1.3.2) expression in prostate (Lin A M, Hershberg R M, Small E J. Immunotherapy for prostate cancer using prostatic acid phosphatase loaded antigen presenting cells. Urol Oncol 2006; 24: 434-41). US2006/0294615 discloses a therapeutic method for treating a mammalian prostate carcinoma comprising the step of administering a therapeutically effective amount of cellular PAP protein to the carcinoma. This and all the other publications referred to herein are incorporated by reference.

There are two forms of PAP, secretory and non-secretory, with different isoelectric points and molecular weights (Vihko P. Human prostatic acid phosphatases: purification of a minor enzyme and comparisons of the enzymes. Invest Urol 1979; 16: 349-52). Only mRNA encoding secretory form is described so far (Vihko P, Virkkunen P, Henttu P, et al. Molecular cloning and sequence analysis of cDNA encoding human prostatic acid phosphatase. FEBS Lett 1988; 236: 275-81). It is suggested to encode also so-called cellular form (Veeramani S, Yuan T C, Chen S J, et al. Cellular prostatic acid phosphatase: a protein tyrosine phosphatase involved in androgen-independent proliferation of prostate cancer. Endocr Relat Cancer 2005; 12: 805-22). Cellular PAP has not been cloned.

It is claimed that PAP has growth-suppressing effect and it is due to its cellular protein tyrosine phosphatase activity. Within cells the activity of PAP is lower in prostate carcinomas than in normal prostates and both PAP mRNA and protein levels are decreased or absent in prostate carcinoma tissue (Hakalahti L, Vihko P, Henttu P, et al. Evaluation of PAP and PSA gene expression in prostatic hyperplasia and prostatic carcinoma using northern-blot analyses, in situ hybridization and immunohistochemical stainings with monoclonal and bispecific antibodies. Int J Cancer 1993; 55: 590-7). U.S. Pat. No. 7,094,533 discloses methods for diagnosing androgen-insensitive prostate carcinomas comprising the step of determining the expression of cellular PAP protein in the prostate carcinoma, a decrease in the expression being indicative of the androgen insensitive nature of the carcinoma.

New results found by the present inventor show that PAP has at least two splicing variants encoding a secretory form and a type I transmembrane (TM) protein (Quintero et al., 2007, Cancer Res 67, 6549-6554), which is in vesicles and membranes and is widely expressed in many non-prostatic cells and tissues.

According to the new results, it is important to highlight the fact that the expression of PAP is not exclusive to prostatic tissue, and this issue has to be considered for the evaluation of unwanted side effects of PAP-based immunotherapy. The present invention suggests TM-PAP to have still unrevealed physiological prostatic and non-prostatic functions.

### SUMMARY OF THE INVENTION

Prostatic acid phosphatase (PAP) is currently evaluated as a target for vaccine immunotherapy of prostate cancer. This is based on the previous knowledge about secretory PAP and its high prostatic expression. Herein a novel PAP spliced variant mRNA encoding a type I transmembrane (TM) protein with the extracellular N-terminal phosphatase domain and the C-terminal endosomal/lysosomal targeting signal (Yxxc.phi.) is described. This TM-PAP is widely expressed in non-prostatic tissues like brain, brown adipose tissue, kidney, liver, lung, muscle, placenta, salivary gland, spleen, thyroid and thymus. TM-PAP is also expressed in fibroblasts, Schwann and LNCaP cells, but not in PC-3 cells. In well-differentiated human prostate cancer tissue specimens the expression of secretory PAP, but not TM-PAP, is significantly decreased. TM-PAP is localized in plasma membrane-endosomal-lysosomal pathway and is co-localized with the lipid raft marker flotillin-1. No cytosolic PAP is detected.

It is concluded that the wide expression of TM-PAP in, for instance, neuronal and muscle tissues, must be taken into account in the design of PAP-based therapy approaches.

Snapin is a protein associated in SNARE complex. “Yeast Two-Hybrid and co-immunoprecipitation was made as in Snapin interaction with the Exo70 subunit of the exocyst and modulates GLUT4 trafficking, J Biol Chem 283, 324-331, 2008” describes the role of Snapin in exocytose. The present inventor has discovered that TM-PAP interacts with Snapin therefore having a role in ecto- and exocytosis. This happens in all the cells wherein the Snapin is expressed. Snapin-mediated exocytosis is very important for the transport of sugar transport channels onto cell membrane. Also TM-PAP, when controlling Snapin, will also take part in this mechanism which controls the ion channels in prostate, kidneys and lungs. Also antigen presentation, immune response, inflammation response and autoimmune response are controlled with this mechanism. This is important for the applications of the present invention especially in prostate, nerve tissue and brains. For example, prostate cancer is a vesicle transport disorder (or vesicular transfer disease as both terms may be used interchangeably). In the vesicle transport disorders the vesicular traffic and release is disturbed. Generally the vesicular traffic is overfunctioning leading for example to overactivity of antigen presentation, immune response, inflammation response or autoimmune response, or overactivity of ion channels in prostate, kidneys and lungs. This may be controlled by TM-PAP.

Exocytosis is also the mechanism of neurotransmitter secretion. TM-PAP is recycling on endosomal-exosomal pathway of the cell. It was surprisingly found that TM-PAP is expressed in a subpopulation of cerebral GABAergic neurons, and mice deficient in TM-PAP show multiple behavioral and neurochemical features of schizophrenia. Herein it is show that TM-PAP is localized presynaptically, and linked to SNARE-associated protein Snapin, a protein involved in synaptic vesicle docking and fusion, and that PAP-deficient mice display altered subcellular distribution of Snapin. It seems that through the mechanism of disturbed exocytosis TM-PAP is involved in the control of GABAergic neurotransmission in the brain and GABAergic neurons, and that PAP deficiency produces a global schizophrenic phenotype.

The TM-PAP knockout/knockdown mouse described herein is a model animal for these disorders. The secreted PAP does not interact with Snapin because the interacting part is the intracellular part not found in secreted PAP. Snapin is an intracellular protein. It was also found out in the present invention that when prostate cancer cells PC-3 were transfected with TM-PAP the growth of the cell was arrested. This does not happen if secreted PAP is used.

It is characteristic for the present invention what is disclosed in the independent claims. Some embodiments of the invention are disclosed in the dependent claims.

The present invention relates to a method for treating vesicle transport disorders involving SNARE complex/Snapin interaction, comprising increasing the level or restoring the activity of transmembrane prostatic acid phosphatase in a patient suffering from said disorder. In one example the present invention relates to a method for treating vesicle transport disorders involving SNARE complex/Snapin interaction, comprising administering a transmembrane prostatic acid phosphatase protein having a transmembrane domain and endosomal/lysosomal targeting signal in the C-terminus, or the C-terminal part thereof having a transmembrane domain and endosomal/lysosomal targeting signal in the C-terminus, to a patient suffering from said disorder.

One aspect of the present invention relates to a novel prostatic acid phosphatase protein having a transmembrane domain and endosomal/lysosomal targeting signal in the C-terminus, or the C-terminal part thereof.

Another aspect of the present invention relates to an isolated DNA molecule encoding said transmembrane prostatic acid phosphatase protein or the C-terminal part thereof.

Still another aspect of the present invention relates to a nucleotide vector containing said DNA molecule.

Still another aspect of the present invention relates to a recombinant host cell containing said nucleotide vector.

Still another aspect of the present invention relates to a pharmaceutical composition containing said transmembrane prostatic acid phosphatase protein or the C-terminal part thereof.

Still another aspect of the present invention relates to said transmembrane prostatic acid phosphatase protein or the C-terminal part thereof for use as medicament.

Still another aspect of the present invention relates to use of said transmembrane prostatic acid phosphatase protein or the C-terminal part thereof for manufacturing medicament for treating disorders such as vesicle transport disorders, such as cancer, for example prostate cancer, prostatitis, respiratory or kidney diseases; inflammatory, immunodefence or autoimmune diseases, lymphoproliferative disorders such as leukemia, bone marrow proliferation, metabolic disorders.

Still another aspect of the present invention relates to a method for delivering drugs, nanoparticles, imaging reagents or other molecules to cells using said transmembrane prostatic acid phosphatase protein or the C-terminal part thereof as a carrier.

Still another aspect of the present invention relates to a method for delivering agents, such as the TM-PAP, or drugs, nanoparticles, imaging reagents or other molecules to cells using an exosome as a carrier.

Still another aspect of the present invention relates to a method for treating disorders such as vesicle transport disorders, such as cancer, for example prostate cancer, lymphoproliferative disorders such as leukemia, bone marrow proliferation, or metabolic disorders, or disorders related to neurotransmitter packing and release, such as disturbed neurotransmission, neuropsychiatric disorder or increased GABAergic transmission, by administering said transmembrane prostatic acid phosphatase protein or the C-terminal part thereof to a patient suffering from said disorder. In one example the vesicle transport disorder is schizophrenia.

Still another aspect of the present invention relates to a method for diagnosing prostate cancer by determining the ratio of TM-PAP and secreted PAP in a tissue or a body fluid.

Still another aspect of the present invention relates to a non-human animal having a disruption in the transmembrane prostatic acid phosphatase gene or regulation thereon resulting in a decrease or absence of the activity or the level of prostatic acid phosphatase. The present inventor has previously described such knockout animal having a disruption in secreted PAP gene in WO06051172A1, which is incorporated herein by reference. At that time there was no knowledge about transmembrane PAP, but the concept and the uses of said animal model can be directly applied also to TM-PAP.

One aspect of the present invention relates to a method for testing and screening for a compound for an therapeutic effect, said method comprising the steps of administering said compound to a cell or a non-human animal having disruption in the transmembrane prostatic acid phosphatase gene or regulation thereof resulting in a decrease or absence of the activity or the level of prostatic acid phosphatase, and determining if said compound substantially restores the unbalanced phosphatidylinositol phosphate signaling pathway related to TM-PAP expression or activity on said cell or said animal, said restoring indicating said compound being therapeutically effective for treating disorders related to unbalanced phosphatidylinositol phosphate signaling pathway. The response of said restoring may be decrease in PI(4,5)P.sub.2 accumulation or decrease in the level of PI(3)P, caused by the recovered PAP activity or level of expression. The term “substantially restoring” as used herein refers to such restoration or normalization of unbalanced PIP signaling pathway, either complete or partial, which has therapeutic value and effect. These methods may be used for investigating diseases and disorders related to TM-PAP, such as vesicle transport disorders, for example prostate hypertrophy, tumors or cancer. Such methods include for example testing and screening of a drug candidate compound or the like.

Another aspect of the current invention relates to methods for treating disorders related to unbalanced phosphatidylinositol phosphate cascade and/or signaling pathway related to transmembrane prostatic acid phosphatase resulting in PI(4,5)P.sub.2 accumulation, increased level of PI(3)P or increased ratio of PI(4,5)P.sub.2/PI(4)P, by administering a patient suffering said disorder a compound which increases the level of expression or activity of TM-PAP or by giving said patient gene therapy which increases the level or restores the activity of TM-PAP.

The present invention also utilizes a knockout/knockdown animal model wherein the transmembrane prostatic acid phosphatase gene in the genome of said animal has been disrupted resulting in a decrease in the activity or the level of transmembrane prostatic acid phosphatase. Said knockout/knockdown animal expresses a reduced level or activity of TM-PAP enzyme in certain cells or tissues or preferably does not express TM-PAP at all.

Also an isolated knockout/knockdown animal or plant cell, such as a prostate cell, may be used. Such cells may be cultured and used to investigate disorders related to TM-PAP and its function. The animals described above may be used as a source of said cells. In one embodiment said cell may be a human cell line, such as one derived from a human cancer cell line.

### BRIEF DESCRIPTION OF THE FIGURES

FIG. 1. Expression and splicing of PAP variants. (A) Total RNA from human, mouse and rat prostate tissue was analyzed for TM-PAP variant by RT-PCR using ATG and TGA containing primers or primer from 3′ side of the stop codon. (B) Exon-intron boundaries of the alternatively spliced intron and deduced amino acids of TM-PAP and secreted PAP. Upper case letters represent exon nucleotides (upper line) and amino acid residues (lower line), and lower case letters represent intron nucleotides of PAP (upper line). The length of spliced region is expressed as by in parentheses. The number of non-presented nucleotides in the last exon and amino acid residues in the C-terminus is marked in parentheses. Conserved 5′ and 3′ splice site nucleotides of introns are marked in bold. * marks a stop codon, (1)-(2) human, (3)-(4) mouse and (5)-(6) rat PAP, TM and secreted variants, respectively. Nucleotide sequence for rat TM-PAP variant was predicted from genome through our own Perl script programs using regular expressions. (C) Alignment of N- and C-termini of TM-PAP isoform with LAP. Identical residues are indicated by white letters in black boxes. Asterisks indicate residues required for lysosomal targeting (Yxxc.phi.). Alignments were performed using Clustal W (Thompson J D, Higgins D G, Gibson T J. CLUSTAL W: improving the sensitivity of progressive multiple sequence alignment through sequence weighting, position-specific gap penalties and weight matrix choice. Nucleic Acids Res 1994; 22: 4673-80) and ESPript (Gouet P, Courcelle E, Stuart D I, Metoz F. ESPript: analysis of multiple sequence alignments in PostScript. Bioinformatics 1999; 15: 305-8) for figures. Secondary structure elements for ESPript were obtained from PHD through PredictProtein server (Rost B, Yachdav G, Liu J. The PredictProtein Server. Nucleic Acids Res 2004; 32: W321-W326). Transmembrane prediction was performed with TMHMM server (Krogh A, Larsson B, von Hejne G, Sonnhammer E L. Predicting transmembrane protein topology with a hidden Markov model: Application to complete genomes. J Mol Biol 2001; 305: 567-80) and signal peptide was predicted using SignalP server (Bendtsen J D, Nielsen H, von Heijne G, Brunak S. Improved prediction of signal peptides: SignalP 3.0. J Mol Biol 2004; 340: 783-95). Gene Bank accession numbers: rat TM-PAP variant DQ826426, mouse TM-PAP variant NM.sub.207668, human TM-PAP variant BC007460, human secreted PAP variant NM.sub.001099, mouse LAP mRNA BC023343, human LAP mRNA BC093010 and human LPAP (lysophosphatidic acid phosphatase) mRNA AB031478. (D) Expression of TM-PAP in different mouse cells and tissues, and in human prostate cancer cells, and BPH and PC patient samples.

FIG. 2. PAP and BMP co-localization in human prostate cancer tissue. (A) PAP was localized in vesicles both in the basal and apical cytoplasm, and strong labeling was observed in the lumen of the glands. PAP showed an almost complete co-localization with BMP(PAP green, BMP red, nuclei in blue, co-localization in yellow). (B) PAP localized in the limiting and internal membranes of apical, electron-lucent vesicles (black arrow). PAP was also observed in the lumen of multi-vesicular endosomes. (C) In addition, PAP labeling was observed in the membranous structures in the lumen. PAP (10 nm gold) co-localized with BMP (5 nm gold, arrowheads). Some BMP labeling was also observed in the endosomes (right, arrowheads). AJ=adherent junction, L=lumen.

FIG. 3. PAP localization in LNCaP and SW10. (A) In LNCaP cells, PAP was detected in caveosome-like structures (.about.100 nm, left), small endosome-like vesicles (.about.65 nm, middle), and in lysosomes (right). In (A, left) gold labeling was enhanced by silver just enough to make the nanoparticles visible (indicated by arrows), to avoid any masking of the structures by the precipitate, whereas in (A, middle and right), a stronger signal was obtained by longer enhancement period. (B) PAP in isolated mouse Schwann cells. PAP was localized in the plasma membrane domains and filopodias.

FIG. 4. (A) TM-PAP localization in sarcolemma of human skeletal muscle detected by immunohistochemistry. (B) Localization of TM-PAP-GFP in plasma membrane and vesicles of PC-3 cells after transfection. Co-localization of PAP with different cell markers in LNCaP cells. (C) PAP co-localized with flotillin-1 in small vesicles and in the plasma membrane. (D) PAP co-localization with LAMP-2. White arrows show co-localization sites (yellow).

FIG. 5. The sequence alignment of the whole amino acid sequences of human, mouse and rat TM-PAPs and corresponding secreted PAPs. Signal peptide and transmembrane regions are marked as in FIG. 1.

FIG. 6. Figures (A)-(C) present the Snapin sequences (highlighted) which were found to interact with TM-PAP with yeast two-hybrid system.

FIG. 7 Development of prostate adenocarcinoma in PAP^{-/-} mouse DLP. (a) H&E stainings of PAP^{+/+} DLPs. Monolayer epithelium with open lumen and thin fibromuscular sheath in animals of different ages. (b) H&E stainings of PAP^{-/-} DLPs. Epithelial hyperplasia was present in DLP of 3-month-old PAP^{-/-} mice. Lumen filled with dysplastic epithelial cells, and mPIN structures were observed in 6-month-old animals. Bulging of epithelial cells into the stroma (black arrows) through loosen fibromuscular sheath and prostate adenocarcinoma in 12-month-old mice. FS: fibromuscular sheath, L: lumen, mL: monolayer epithelium. Six to eight PAP^{+/+} and PAP^{-/-} mice were analyzed respectively per each age group. Scale bar is 100 μm.

FIG. 8 PAP^{-/-} mice develop prostate adenocarcinoma. (a) Hematoxylin-eosin stainings of PAP^{+/+} and PAP^{-/-} DLPs from 12- and 24-month-old animals. Upper row, 12-month-old: left, monolayer epithelium and open lumen. Middle and right, glands filled with epithelial cells, dyscohesion, double nuclei (white arrows). Middle row: left, microinvasions (black arrow) and cribriform structures (white arrowhead). Middle, blood vessels among neoplastic epithelial cells (*), invading epithelial cells, prominent nucleoli (black arrow). Right, clusters of cells with hyperchromatic nucleus (black arrowhead). Lower row, 24-month-old: left, regular glandular structures with open lumen. Middle, prostate adenocarcinoma and fibrotic stroma with cellular invasions. Right, amplification showing microinvasion of cells into the stroma and bulging (black arrows). Six to eight prostates were studied in each mouse group at both ages. Scale bars: 100 μm (b) Smooth muscle actin (SMA) immunohistochemistry in 12-month-old mice. Upper row: left, PAP^{+/+} DLP with monolayer epithelium and open lumen. Middle, PAP^{-/-} DLP showing broken fibromuscular sheath (white arrows), bulging of epithelial cells. Right, prostate adenocarcinoma, transformed cells (black arrow). Lower row: Adenocarcinoma of PAP^{-/-} AP. Left, monolayer epithelium of PAP^{+/+} AP. Middle and right, invasive prostate adenocarcinoma (black arrows), inflammatory cell clusters (black arrowhead). Four PAP^{+/+} and PAP^{-/-} mice were analyzed respectively per each age group. Scale bars: 100 μm. mL: monolayer epithelium, ML: multilayer epithelium, SM: smooth muscle. (c) Pan cytokeratin immunohistochemistry of DLP from 12-month-old animals. Left, monolayer epithelium with open lumen in PAP^{+/+} mouse. Middle: epithelial cells crowding into the lumen and stroma of PAP^{-/-} DLP. Epithelial cells with double nuclei. Right, clusters of malignant epithelial cells in the fibrotic stroma. Three animals per group were analyzed. Scale bars: 100 μm. (d) Ultrastructural changes in DLP of 3-month-old mice. Upper row: left, PAP^{+/+} DLP, apical secretory epithelium. Middle and right, numerous microvesicles present in the apical region of PAP^{-/-} DLP and secreted into lumen. Scale bars: 1,000 nm. Lower row: left (scale bar: 500 nm) and middle (scale bar: 200 nm), lamellar body-like structures (*) inside the epithelial cell and released into the lumen (*). Microvilli (black arrowheads), electron-lucent microvesicles (black arrow) in the lumen. Right, electron-dense (white arrow) and electron-lucent (black arrow) microvesicles, and membranous structures in the lumen. Three animals per group were analyzed. Scale bar: 1,000 nm. AJ: adherens junction, L: lumen, MV: microvesicle, MVE: multivesicular endosome, SV: secretory vesicle.

FIG. 9 The ultrastructures of mouse and human prostate adenocarcinomas have common features. (a) PAP^{+/+} DLP from 2-month-old animal with monolayer epithelium, regular basement membrane (BM) and apical secretion. (b) PAP^{-/-} DLP at 3 months with irregular BM and numerous vacuoles. (c) Basal lysosomes in PAP^{-/-} DLP. (d) Multilayer epithelium with hyperchromatic nuclei and multiple nucleoli. Pseudolumens formed as a result of the growing and fusion of the epithelium of PAP^{-/-} DLP at 12 months. (e) Amplification of (d). Invaginations of BM (white arrows) into the epithelium. (f) Microvesicles secreted into basolateral intercellular space of PAP^{-/-} DLP. (g) Human prostate cancer, pseudolumens with membranous material, numerous apical multivesicular endosomes. (h) Pseudolumen, nonpolaric secretion, invasion to BM. (i) Membranous vesicles in pseudolumens, broken BM. Three to four PAP^{+/+} and PAP^{-/-} mice were analyzed respectively per each age group. Scale bars are 2000 nm. AJ: adherens junction, BM: basement membrane, L: lumen, Ly: lysosome, MV: microvesicles, PL: pseudolumen.

FIG. 10 Co-localization and interaction of TMPAP with snapin in the lamellipodia. Co-localization (white) of TMPAP (green) with snapin (magenta) was observed in the vesicles and lamellipodia of the LNCaP pMX TMPAP cells. Arrows mark the co-localization points in the upper panel. Lower panel showing the lamellipodia region is an amplification of the area marked with a box in the upper panel (left). The scale bar for pictures in the upper panel is 20 μm and for the lower panel is 3 μm.

FIG. 11 Proposed model for TMPAP-snapin interaction. The snapin protein can be found either in the cytosol or interacting with SNARE components or other proteins, such as TMPAP, at the vesicle or plasma membrane. TMPAP synthesized in the endoplasmic reticulum is transported in vesicles to the plasma membrane through the trans-Golgi network (TGN). After the vesicle docking and fusion events leading to release of vesicle content, TMPAP inserted in plasma membrane exerts its phosphatase function over adenosine monophosphate (AMP). The resulting product adenosine (Ado) activates the adenosine receptors A1 or A3 with Gαi (inhibitory G-protein β-subunit) specificity leading to the inhibition of adenylate cyclase (AC) activity, and A2 adenosine receptors with Gas (stimulatory G-protein α-subunit) producing the stimulation of AC activity. Activated AC produces cyclic adenosine monophosphate (cAMP), which activates protein kinase A (PKA) responsible for the phosphorylation of snapin. The turnover is completed by clathrin-mediated endocytosis of SNARE components and TMPAP for recycling and degradation in lysosomes via the endosomal-lysosomal pathway. From early endosomes, the cargo can be sorted to late endosomes or to multivesicular endosomes (MVE), which can follow the route leading to exosome release. Additional dephosphorylation events by TMPAP can occur while trafficking between different compartments. From late endosomes, TMPAP can go to lysosomes or back to TGN via the retrograde pathway. TMPAP: transmembrane prostatic acid phosphatase. ATP: adenosine triphosphate, ADP: adenosine diphosphate, AMP: adenosine monophosphate, Ado: adenosine, TGN: trans-Golgi network, P: phosphate group, AP-2: adaptor protein complex 2, ADORA: adenosine receptor A (types A1, A2 and A3), AC: adenylate cyclase, Gαs, Gαi, Gβ, Gγ: G-protein subunits, VDCC: Voltage-gated calcium channel.

FIG. 12 T2-weighted images from WT (B, D) and PAP^{-/-} mice (A, C), and plot of lateral ventricle volumes for WT and PAP^{-/-} mice (E). Lateral ventricles (right and left ventricles) volume increase significantly (** p<0.01) in both the young PAP^{-/-} mice (2 months) and old PAP^{-/-} mice (12 months) compare to corresponding WT mice. The data is expressed as mean+S.E.M.

### DETAILED DESCRIPTION OF THE INVENTION

Novel PAP Isoform is Encoded by Alternative Splice Variant (Quintero et al. (Cancer Res 2007; 67: (14) 6549-6554)).

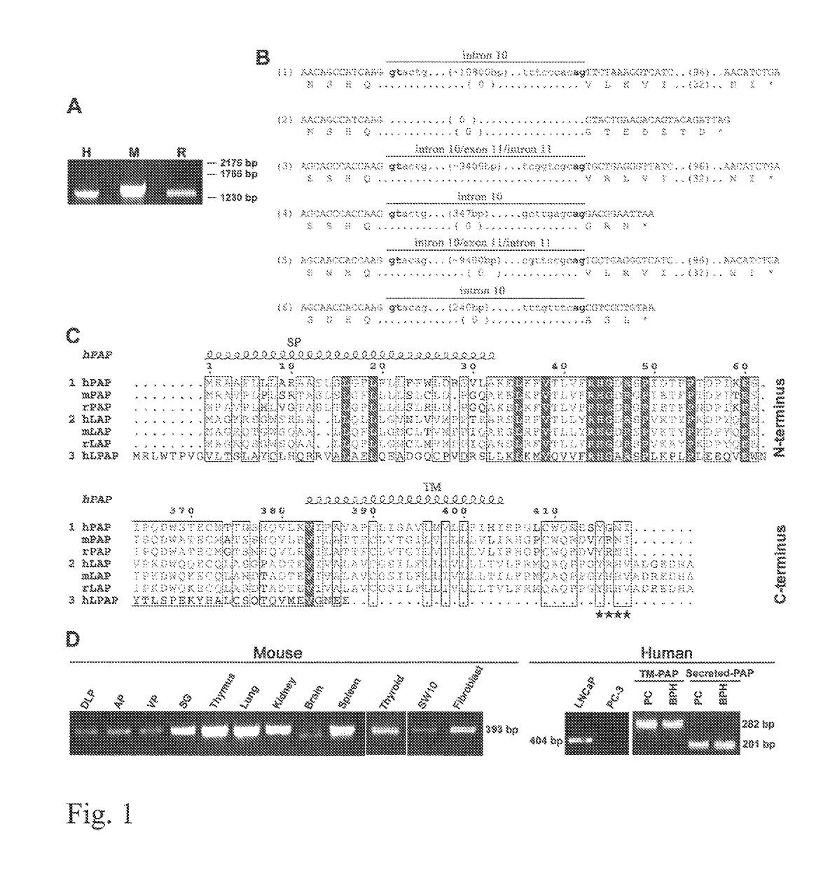

To generate human, mouse and rat TM-PAP variants by RT-PCR (FIG. 1A) total RNA was used. Rat and human PCR products were cloned and sequenced. Sequences were highly homologous with cDNA of mouse TM-PAP variant. The rat cDNA (GenBank accession number DQ826426), showed 91% and 81% identities with reported cDNA sequences of mouse (GeneBank accession number NM.sub.-207668) and human PAP, respectively. Comparison of the exon-intron junction suggests that rat, mouse and human PAP variants are derived by alternative splicing. Position of the splicing of the 10.sup.th intron in the rat, mouse and human gene is similar at the end of the 10.sup.th exon. In the rat and mouse gene, the splicing of the 10.sup.th intron results in the secreted variant mRNA and in human in the TM variant. The splicing of the 10.sup.th intron, the 11.sup.th exon and the 11.sup.th intron in the rat and mouse gene yields the TM variant mRNA. In the human secreted variant mRNA, the open reading frame (ORF) continues over the splicing site until the stop codon (FIG. 1B).

The deduced 417-amino acid sequence showed 89% and 95% homologies with human and mouse PAP, respectively. The N-terminus of PAP has a 32 amino acids long signal peptide. The C-terminus of TM-PAP isoform contains the TM domain of 22 amino acid residues and an endosomal/lysosomal targeting Y[G/R]NI sequence separated by 10 amino acids from TM domain (FIG. 1C). The relative location of signal and TM peptides determines the topology of the TM-PAP isoform as a type I transmembrane protein. The present tyrosine signal Y[G/R]NI is similar to the lysosomal targeting sequence Yxx.phi., where x can be any amino acid but tends to be hydrophilic and .phi. is a hydrophobic residue. As reviewed by Bonifacino and Traub (Bonifacino J S, Traub L M. Signals for sorting of transmembrane proteins to endosomes and lysosomes. Annu Rev Biochem 2003; 72: 395-447) this is evidence for lysosomal targeting. Moreover, the C-terminal residue of PAP, isoleucine, is the same as in LAMP-1, a type I TM protein known to be targeted to lysosomes. In conclusion, TM-PAP isoform can be efficiently internalized from plasma membrane due to the presence of the Yxxl motif and delivered to lysosomes through the indirect endosomal pathway as confirmed by our observations. Alignment of human, mouse and rat TM-PAP and lysosomal acid phosphatase (LAP) reveals similar TM domain, and lysosomal targeting sequence (FIG. 1C). Human LAP presents two cleavage sites for two different proteases. When C-terminal ends of hLAP and hPAP transmembrane isoform are compared, there is no identity of amino acids at the protease cleavage site or at the neighboring residues. Specificity requirements of these proteases suggest that TM-PAP is not a suitable substrate for them and it is possible that TM-PAP is recycling from lysosomes to plasma membrane.

TM-PAP is Widely Expressed

Based on the contradictory statements present in the literature about the tissue-specific expression of PAP, the expression of this novel splicing variant in both human and mouse was analyzed. RT-PCR analyses showed that the TM-PAP variant was widely expressed in mouse tissues e.g. in prostate lobes, salivary gland, thymus, lung, kidney, brain, spleen, thyroid and mouse Schwann and fibroblast cells (FIG. 1D). Expression of the PAP secreted variant was clearly detected in salivary gland, thymus and thyroid (data not shown). Secretory and TM-PAP variants were present in LNCaP cells, but they were not expressed in PC-3 cells (FIG. 1D).

The Ratio of PAP Isoforms is Changed in Prostate Cancer

Since immunohistochemically, PAP shows low expression in prostate carcinoma cells and tissues although in the circulation of prostate cancer patients its concentration may be elevated, PAP expression in prostate was further studied. Both PAP variants were present in human BPH and well-differentiated PC specimens (FIG. 1D). The abundance of PAP variants in BPH (n=27) and PC (n=19) specimens was determined using quantitative real-time PCR. Values of TM-PAP mRNA variant were (mean.+−.SD) 0.5.+−.0.26 in BPH and 0.45.+−.0.34 in PC, and values of PAP mRNA secreted variant were 0.60.+−.0.3 in BPH and 0.31.+−.0.33 in PC. Both variants were expressed in all specimens, but expression of the PAP mRNA secreted variant was significantly decreased in PC(P<0.05).

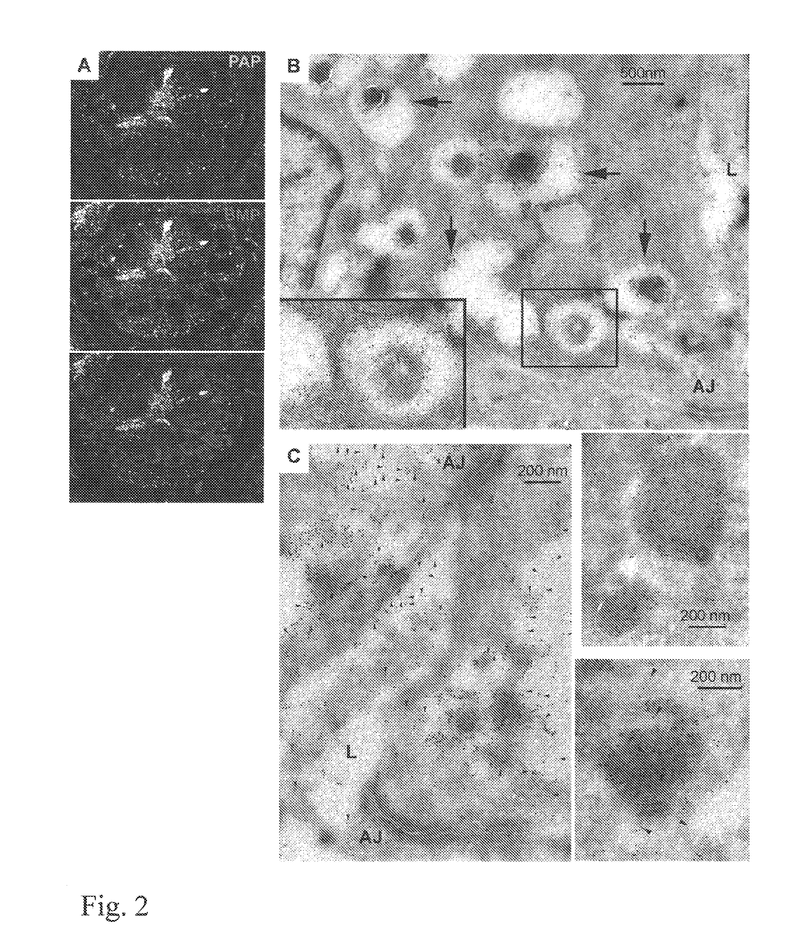

Immunofluorescence and electron microscopy were used to further investigate the localization of PAP in prostate cancer tissue. Immunofluorescence revealed that PAP is localized in vesicles located both in the basal and apical cytoplasm (FIG. 2A, PAP). In addition, strong labeling was observed in the lumen of the glands (FIG. 2A, PAP). PAP showed an almost complete colocalization with BMP (bis(monoacylglycero)phosphate) (FIG. 2A, BMP), a lipid highly enriched in late endosomes and multivesicular bodies (MVB) (FIG. 2A, merge). IEM was used to study the localization of PAP at higher resolution. PAP localized in the limiting and internal membranes of apical, electron-lucent vesicles (FIG. 2B, up), as well as in the internal membranes of MVBs (FIG. 2B, down). In agreement with the immunofluorescence labeling, PAP co-localized with BMP in all these structures (FIG. 2C, right), and PAP was observed in the membranous structures in the gland lumen (FIG. 2B, left). This suggests the luminal material may originate from PAP and BMP containing vesicles which have fused with the plasma membrane and emptied their contents into the lumen. These observations suggest that at least some luminal PAP is associated with intraendosomal membrane structures. Normally, the internal vesicles of the MVB are destined for degradation and are released into the lysosomal lumen. The residual membrane of the MVB is believed to retain those proteins which will escape degradation. MVB can also recycle to the plasma membrane and release their contents (exosomes) in vivo into the circulation.

PAP is in Vesicles and Membranes

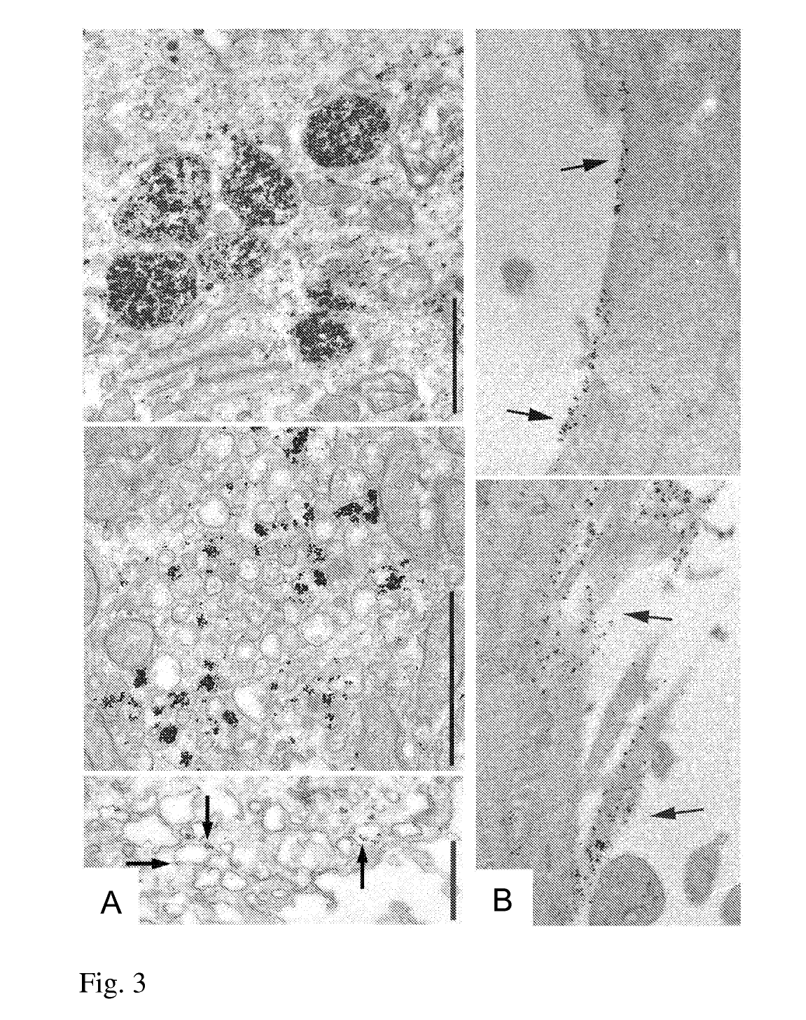

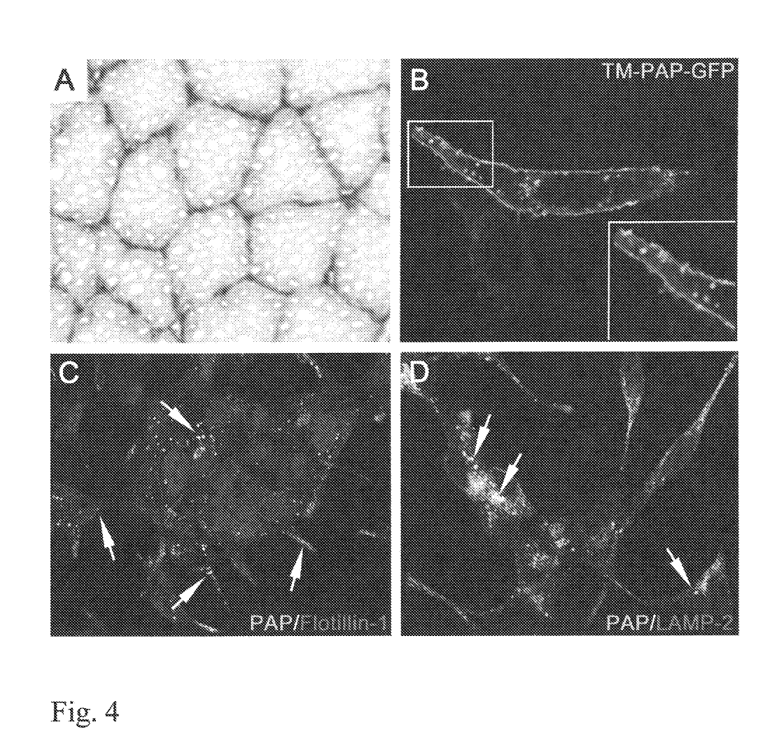

Similar pattern of distribution was detected in LNCaP cells, with a localization of PAP in endosome-like vesicles and lysosomes (FIG. 3A). The analysis of mouse Schwann cells showed PAP in the plasma membrane segments and filopodia-like structures (FIG. 3B). In the human skeletal muscle fibers, PAP expression was present in the sarcolemma (FIG. 4A).

The subcellular localization of TM-PAP-GFP in transfected PC-3 cells that do not express PAP endogenously was examined. Subcellular localization of TM-PAP-GFP in PC-3 cells was analyzed by confocal microscopy. TM-PAP-GFP was seen on the plasma membrane as expected and also observed in intracellular vesicles (FIG. 4B). In transfected control cells with an empty EGFP-N3 vector the signal was evenly distributed throughout the cells.

The co-localization of endogenous PAP with flotillin-1, which is associated with membrane lipid raft, was studied further and it showed that both proteins colocalize in plasma membrane and intracellular vesicles (FIG. 4C). Flotillins have been implicated in signaling, regulation of actin cytoskeleton and in membrane transport processes.

Considering the alignment results showing that PAP contains the lysosomal targeting sequence Yxx.phi., the co-localization of PAP with a lysosomal associated type I membrane protein, LAMP-2 (FIG. 4D), was studied. The findings showed a clear co-localization between these proteins.

In conclusion, there are two widely expressed isoforms for PAP, a secreted and a transmembrane one. The importance of these two isoforms in prostate cancer development remains to be addressed, but the presence of a novel TM-PAP isoform in plasma membrane would suggest it to be involved in crucial cellular functions.

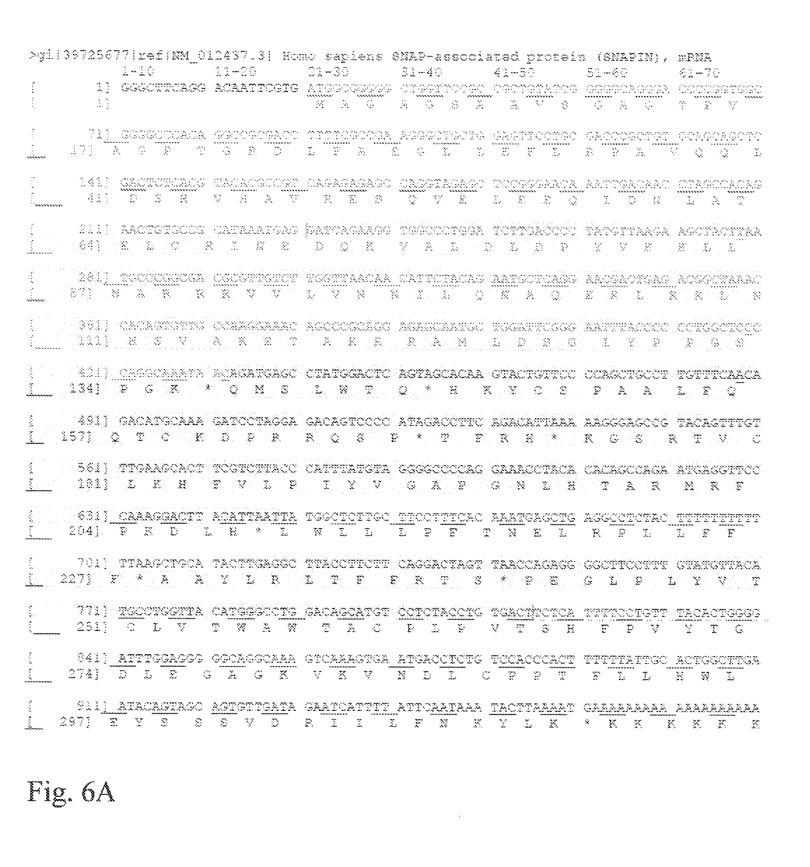

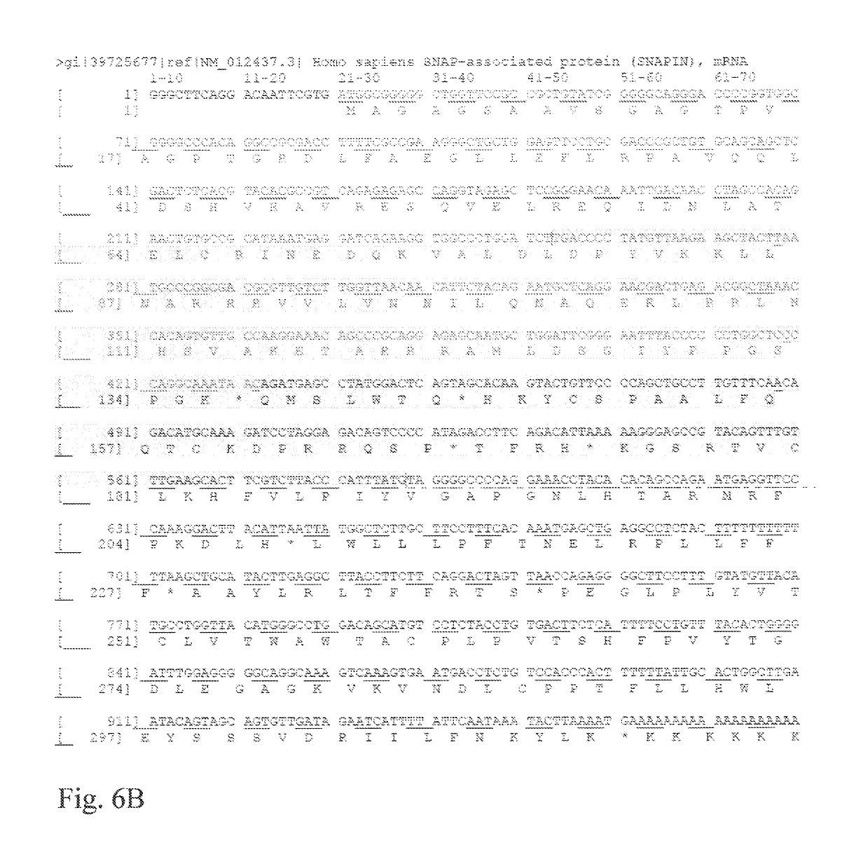

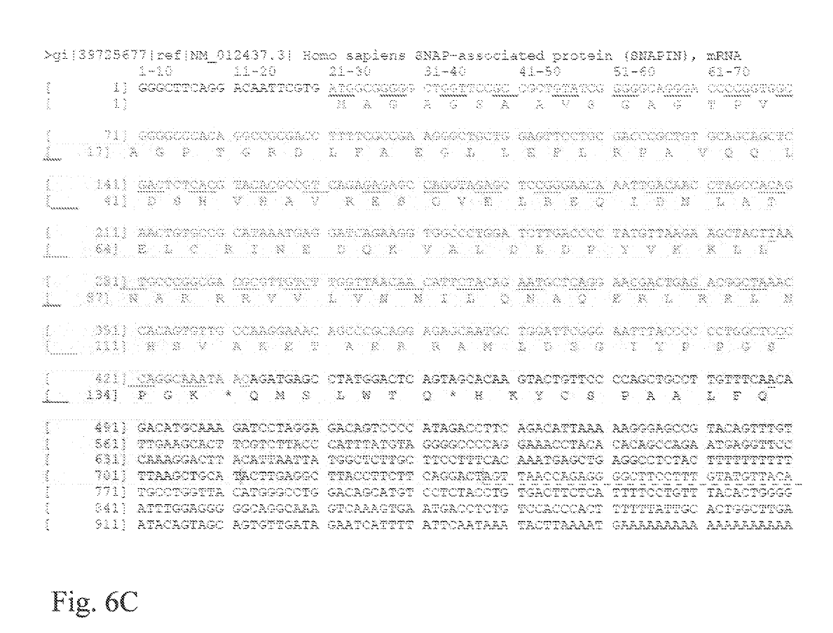

The yeast two-hybrid system was used to find out proteins interacting with TM-PAP. The yeast two-hybrid and co-immunoprecipitation was made as in “Snapin interacts with the Exo70 subunit of the Exocyst and modulates GLUT4 trafficking”, J Biol Chem 283, 324-331, 2008. The only finding was Snapin (FIG. 6 (A)-(C)), which is one of the proteins of exocytose mechanism. Therefore also TM-PAP is one part of the controlled secretion i.e. exocytose mechanism. In the microarray the most important group presented is the neurotransmission secretion mechanism. This mechanism is also in prostate and disruption thereof causes a change in the cell polarity. The apical microvilli will disappear, the ion channels, especially Cl and aquaporin channels, are downregulated, basal secretion decreases and cancer develops. Snapin is required together with TM-PAP in both the exocytosis and the endocytosis. TM-PAP will increase the production of cAMP and therefore also secretion. The regulation is via GPCR.

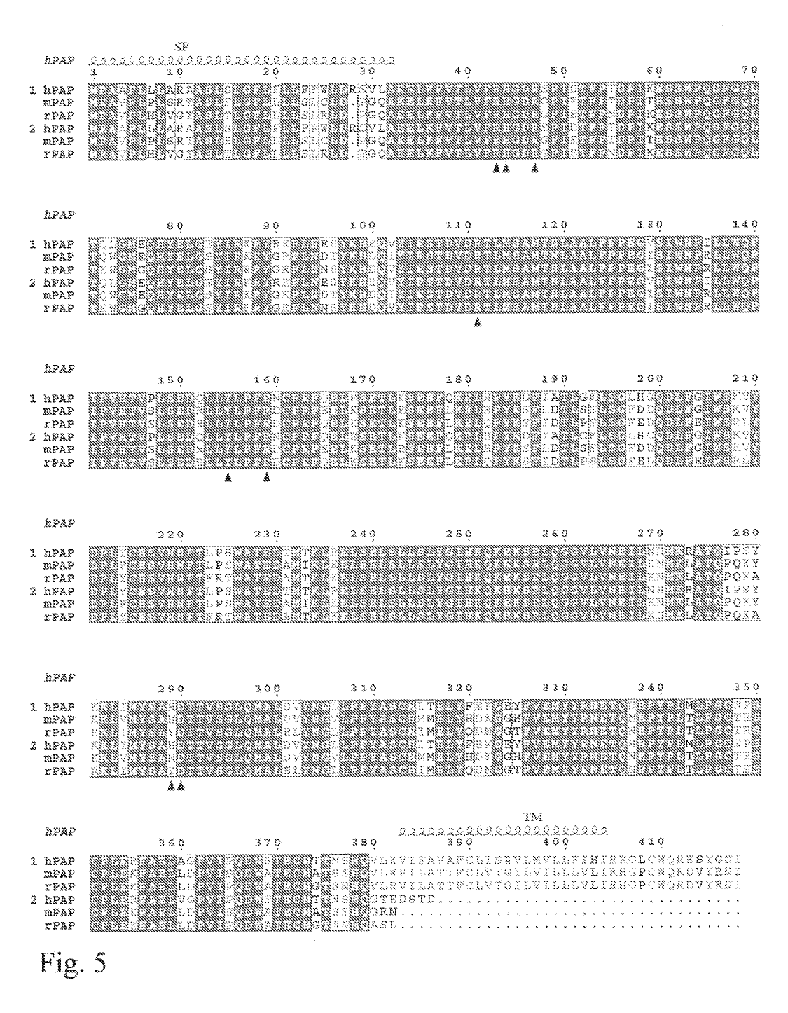

Thus, the present invention relates to a prostatic acid phosphatase protein having a transmembrane domain in the C-terminus, or the C-terminal part thereof. This protein may be used in any of the applications described herein. In one embodiment said transmembrane prostatic acid phosphatase protein is a human prostatic acid phosphatase protein. In still one embodiment said TM-PAP contains the amino acid sequence of SEQ ID NO: 1 or is a C-terminal fragment or a homologue thereof. Also the mouse and rat counterparts as shown in FIG. 5 are included. The about 25 kDa C-terminal part may also be cleaved off and used as a separate functional domain. The C-terminal part may be easily determined e.g. from FIG. 5 wherein several TM-PAP and secreted PAP sequences are aligned. The C-terminal part may contain e.g. the transmembrane region marked (amino acids 383-404). In one example said homologue of the TM-PAP has at least 80% homology at amino acid level, for example at least 80% homology, such as at least 95% homology, to the amino acid sequence of SEQ ID NO: 1, and has active transmembrane domain and the endosomal/lysosomal targeting signal part in the C-terminus. In one example said PAP part has at least 80% homology at amino acid level, for example at least 80% homology, such as at least 95% homology, to the amino acids 1-382 of SEQ ID NO: 1.

In one example the region containing the transmembrane domain and the endosomal/lysosomal targeting signal in the C-terminus (C-terminal end) has the amino acid sequence of amino acids 383-418 of SEQ NO: 1 as shown in FIG. 5, or is a homologue thereof, for example a homologue having at least 80% homology at amino acid level, for example at least 80% homology, such as at least 95% homology. The C-terminal part may contain any of the sequences aligned in FIG. 5 (human, mouse or rat), such as said transmembrane region (amino acids 383-404), endosomal/lysosomal targeting signal region (amino acids 405-418), or both, or it may be a consensus sequence thereof. The length of the transmembrane domain and the endosomal/lysosomal targeting signal part at the C-terminal end may be about 25 kDa or about 21 amino acids. In one example such part may be combined recombinantly with any protein having PAP activity. In one example the TM-PAP as whole may be derived from a naturally occurring polypeptide.

TM-PAP may act in receptor transduction pathway together with G-protein coupled receptor (GPRC). The expression of RGSL2, a GPCR regulator, will increase greatly if PAP is knocked out or down. This fits to G(q/11) G-protein-mediated signaling. The secreted PAP may also regulate the extracellular part of TM-PAP, e.g. the binding of ligands to it, because the extracellular N-terminal part of TM-PAP is almost identical to the secreted PAP.

It was also discovered, that the knockout/knockdown of PAP induces RanGTPase and leads to increased and erroneous mitosis. This explains the prostate cancer observed in mice. Thus, PAP is a mitosis regulating factor. Therefore it may be used to treat any excess cell proliferation such as cancer, e.g. prostate cancer, lymphoproliferative disorders such as leukemia, bone marrow proliferation or other disorders.

One embodiment of the present invention relates to a nucleic acid molecule, such as a DNA or RNA molecule, encoding said TM-PAP of the invention or the C-terminal part thereof. Because of the degeneracy of the genetic code there are a number of different nucleic acid sequences encoding the TM-PAP of the invention. All such nucleic acid variants are in the scope of the present invention. Preferably said nucleic acid molecule is a DNA molecule.

One embodiment of the present invention relates to a replicable vector containing the nucleic acid molecule described above in operative association with an expression control sequence thereof. Such vector may be used for producing recombinant TM-PAP or the C-terminal part thereof of the present invention in a suitable host system.

The nucleic acid encoding the TM-PAP or the C-terminal part thereof of the invention may be inserted into said replicable vector for cloning or for expression. Various vectors are publicly available. The vector may, for example, be in the form of a plasmid, cosmid, viral particle or phage. The appropriate nucleic acid sequence may be inserted into the vector by a variety of procedures. In general, DNA is inserted into an appropriate restriction endonuclease site(s) using techniques well known in the art. Vector components may include for example one or more signal sequence(s), an origin of replication, one or more marker genes, an enhancer element, a promoter, and a transcription termination sequence. Construction of such suitable vectors containing one or more of these components employs standard ligation techniques which are well-known to a person skilled in the art.

Generally said TM-PAP or the C-terminal part thereof may be produced recombinantly by expressing in any suitable host cell, such as in a bacterial host cell. Such methods are well-known in the art and they are described in literature. It is essential that the protein is folded properly during the expression and it contains the necessary post-translational modifications.

One embodiment of the present invention provides the TM-PAP or the C-terminal part thereof of the invention for use as medicament. Still one embodiment of the present invention provides a pharmaceutical composition containing the TM-PAP or the C-terminal part thereof of the invention. Said composition may contain also other substances, such as pharmaceutically acceptable carriers, excipients or other pharmaceutical or therapeutical agents. Such compositions or formulations are well known to a person skilled in the art. One example of such medicament or pharmaceutical composition is a vaccine for any vesicle transport disorder, such as cancer, for example prostate cancer. Then medicament may be applied for example using methods disclosed in US2006/0294615, wherein the TM-PAP or the C-terminal part thereof may be in a liposome, which may be comprised of lipofectin, or the TM-PAP or the C-terminal part thereof is administered as coupled to a monoclonal antibody, which may be immunologically specific to a cancer cell, such as human prostate cancer cell.

One embodiment of the present invention provides the use of the prostatic acid phosphatase protein or the C-terminal part thereof of the invention for manufacturing medicament for treating vesicle transport disorders, such as cancer, for example prostate cancer, or other metabolic disorders related to e.g. glucose or lipid metabolism.

One embodiment of the present invention provides a method for treating vesicle transport disorders involving SNARE complex/Snapin interaction, comprising increasing the level or restoring the activity of transmembrane prostatic acid phosphatase, in a patient suffering from said disorder. “Increasing the level of TM-PAP” may refer for example to administration of TM-PAP or a C-terminal part thereof, or it may refer to induction of TM-PAP, such as induction of the expression (transcription or translation) thereof by administering an agent capable of inducing the expression, or to induction of the enzyme activity, for example by administering an activator. It may also refer to administration carried out by using gene therapy. “Restoring the activity of TM-PAP” may refer to an action, wherein a decreased activity of TM-PAP, when compared to a normal or “healthy” activity, is brought to the normal level or close to the normal level.

One embodiment of the present invention provides a method for treating vesicle transport disorders, such as metabolic disorders related to e.g. glucose or lipid metabolism by administering the TM-PAP or the C-terminal part thereof of the invention to a patient suffering from said disorder. One embodiment of the present invention provides a method for treating vesicle transport disorders related to cancer by administering the TM-PAP or the C-terminal part thereof of the invention to a patient suffering from said disorder, such as cancer, such as prostate cancer. TM-PAP may be administered by any suitable means, for example by methods disclosed in US2006/0294615, such as by administering a nucleic acid comprising the coding sequence of TM-PAP to the carcinoma and allowing the coding sequence to be expressed in the carcinoma, or it may be administered as coupled to a monoclonal antibody, which may be immunologically specific to the cancer cell. Besides any cancer cells, the administration using any routes may have also other suitable targets involving SNARE complex/Snapin interaction as described herein, such as neurons.

“Treatment” as used herein refers to various modes of any action which aims for curing, ameliorating or preventing the disorder, disease or medical condition of interest or symptoms thereof, or to improve the condition of a patient suffering from said disease or disorder. A “therapeutically effective amount” of the pharmaceutical composition, medicament or therapeutically effective agent, such as TM-PAP or other agent described herein or a composition thereof, is a quantity of the medicament, composition or agent provided by a particular route of administration and at a particular dosing regimen, that is sufficient to achieve a desired therapeutic and/or prophylactic effect. For example, an amount that results in the prevention of or a decrease in the symptoms associated with a disease that is being treated. The amount of the pharmaceutical composition administered to the subject will depend on the type and severity of the disease, the amenability of the disorder to respond to the medicament, and on the characteristics of the individual and their metabolic ability to respond to the medicament to produce in vivo a response to the treatment. The skilled artisan will be able to determine appropriate dosages depending on these and other factors.

For the purpose of this invention, the medicament or a pharmaceutically acceptable salt or derivative thereof may be administered by various routes and as various pharmaceutical forms well known in the art. Examples of suitable administration forms include oral formulations, topical formulations, parenteral injections including intravenous, intramuscular, intradermal and subcutaneous injections, and transdermal or rectal formulations.

Suitable oral formulations include e.g. tablets, lozenges or capsules prepared by conventional means with pharmaceutically acceptable excipients such as binding agents (e.g. pregelatinized maize starch, polyvinylpyrrolidone or hydroxypropyl methyl cellulose), fillers (e.g. lactose, microcrystalline cellulose or calcium hydrogen phosphate), lubricants (e.g. magnesium stearate, talc or silica), disintegrants (e.g. potato starch or sodium starch glycolate) or wetting agents (e.g. sodium lauryl sulphate). The tablets may be coated by methods well known in the art. Liquid preparations for oral administration may take the form of for example solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats), emulsifying agents (e.g. lecithin or acacia), non-aqueous vehicles (e.g. almond oil, oily esters, ethyl alcohol or fractionated vegetable oils) and preservatives (e.g. methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

Suitable formulations for parenteral administration include e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g. sterile pyrogen-free water, before use.

Still one embodiment of the present invention provides a method for treating vesicle transport disorders such as metabolic disorders related to e.g. glucose or lipid metabolism by induction of TM-PAP to restore the physiological condition. Still one embodiment of the present invention provides a method for treating vesicle transport disorders such as cancer by induction of TM-PAP to restore the physiological condition. In one example the increasing the level or restoring the activity transmembrane prostatic acid phosphatase protein comprises administering to the patient suffering from said disorder an agent capable of inducing, such as initiating or enhancing, the expression of the transmembrane prostatic acid phosphatase protein. In one example the increasing the level or restoring the activity transmembrane prostatic acid phosphatase protein comprises administering to the patient suffering from said disorder an enzyme activator capable of activating the transmembrane prostatic acid phosphatase.

One embodiment of the present invention provides a method for delivering drugs, nanoparticles, imaging reagents or other useful molecules to cells using said transmembrane prostatic acid phosphatase protein as a carrier or vehicle. For example the drugs nanoparticles, imaging reagents or other useful molecules may be attached to an antibody binding TM-PAP which then finds its way to the appropriate cells. The secreted form of PAP is not able to do this. In one embodiment the antibody binds the extracellular part of TM-PAP. The antibody may also be labeled with any suitable detectable label, such as radioactive or fluorescent label, and it can be used for example to detect metastases. In one embodiment nanoparticles containing drugs have such antibody on their surface.

One embodiment of the present invention provides a method for delivering agents, such as the TM-PAP, or other agents, such as drugs, nanoparticles, imaging reagents or other useful molecules to cells using an exosome as a carrier. Exosomes are living cell organelles which may be considered as nanoparticles because of their size. Exosomes are generally 30 to 90 nm vesicles secreted by a wide range of mammalian cell types. They are larger than the LDL that has a size of 22 nm, but much smaller than a red blood cell, an erythrocyte. Exosomes are released from the cell when multivesicular bodies fuse with the plasma membrane. Exosomes contain various molecular constituents of their cell of origin, including proteins and RNA. Although the exosomal protein composition varies with the cell and tissue of origin, most exosomes contain an evolutionary-conserved common set of protein molecules. Exosomes are also referred to as microvesicles, epididimosomes, argosomes, exosome-like vesicles, microparticles, promininosomes, prostasomes, dexosomes, texosomes, dex, tex, archeosomes and oncosomes (Wikipedia).

One embodiment of the present invention provides a method for diagnosing vesicle transport disorders, such as cancer, such as prostate carcinoma, by quantitating TM-PAP and secreted PAP in a tissue and determining the ratio thereof. As the expression of secreted PAP variant mRNA is decreased in cancer, the elevated ratio of TM-PAP:secreted PAP may indicate the cancer. The ratio may be determined from a tissue sample by any suitable means known for a person skilled in the art enabling the quantitation of TM-PAP and secreted PAP, such as quantitative real-time PCR. U.S. Pat. No. 7,094,533 discloses methods for diagnosing androgen-insensitive prostate carcinomas. In said methods the cellular PAP protein is quantified by an antibody immunologically specific to the cellular PAP or the concentration of cellular PAP mRNA is quantified by PCR, Northern or Southern blot. Said methods may be applied also for quantifying the ratio of TM-PAP and secreted PAP.

The diagnosis may be carried out by detecting exosomes, which are cellular organelles, nanovesicles, carrying proteins, peptides, lipids, DNA, RNA and miRNA. They are endosomes of multivesicular bodies and secreted from cells as exosomes. Flotillin-1 and LAMP-2 are general exosome markers but TM-PAP is a specific exosome marker. TM-PAP is in exosomes as a transmembrane protein and N-terminal acid phosphatase part is on outer surface of the vesicle. The exosomes may be detected from any suitable body fluid, such as blood, saliva, urine, or cerebrospinal fluid, whereto the exosomes are secreted.

One example provides a quantitative immunoassay for body fluid secretory PAP and exosomal TM-PAP.

Exosomes are stable and can be fractionated from body fluids with known methods. For quantification of secretory PAP from supernatant and TM-PAP from exosomes of pellet, body fluid is fractionated by different methods known in the art, such as ultracentrifugation. For example sandwich assay (TR-IFMA) may be used. Required PAP and antibodies may have been made and tested. Also qRT-PCR can be used.

In the method the ratio sPAP/TM-PAP is calculated. For example in prostate cancer the ratio is decreased when compared to normal/healthy ratio. Generally a decreased ratio of sPAP to TM-PAP indicates a vesicle transport disorder, such as cancer or any other disorder described herein.

In vesicular traffic diseases the amount of exosomes in general (TM-PAP negative exosomes) is increased but the amount of TM-PAP positive exosomes is decreased. Flotillin-1 and LAMP-2 may be used to indicate the amount of exosomes in general. In one example the amount of TM-PAP and Flotillin-1 exosomes are determined and the ratio TM-PAP/Flotillin-1 exosomes is calculated. In another example when other general exosomes markers CD13 and CD142 are used and determined, the ratio TM-PAP/CD13/CD142 is calculated. The decreased ratio of TM-PAP positive exosomes to a general exosomes marker (exosomes in general) indicates the presence of a vesicle transport disorder.

“Knockout” as used herein refers to a process of purposely removing a particular gene or trait from an organism or cell. Generally a knockout is a site-specific integration that usually deletes an essential part of a gene of interest. Methods of making knockouts are generally known in the art, for example microinjection and targeted mutation methods. The knockouts of the present invention may be done with any known knockout methods, such as any heritable modifications of the PAP gene, as long as they result to substantial disruption of at least part of the PAP gene or an expression regulation region thereof resulting in substantial disruption of the original function of expressed PAP enzyme or the original function of PAP gene. In one embodiment the knockout is done by replacing a part of the PAP gene with an external nucleic acid molecule and introducing this modified gene into the genome of the animal to replace the original PAP gene. The knockout may also be done by removing the whole gene of interest.

Gene “knockdown” refers to techniques by which the expression of one or more of an organism's genes is reduced, either through genetic modification (a change in the DNA of one of the organism's chromosomes) or by treatment with a reagent such as a short DNA or RNA oligonucleotide with a sequence complementary to either an mRNA transcript or a gene. If genetic modification of DNA is done, the result is a “knockdown organism” (Wikipedia).

The terms “substantially disrupted” or “substantially reduced” are herein intended to mean that substantially lower amount of normal PAP gene product is produced in cells or in organism when compared to normal cells or organisms. Preferably such lower amount refers to essentially undetectable amount of normal gene product. This type of mutation is generally called as a “null mutation” and an animal carrying such a mutation is also referred to as a “knockout animal”. Correspondingly, when an animal cell is used the cell carrying such a mutation is referred to as “knockout cell”.

The “decrease in activity” of the PAP as used herein refers to a decrease of total enzyme activity of said disrupted or substantially disrupted PAP gene product in certain cells. Said decrease in activity may be partial or the activity may be totally lost and it is sufficient to cause disorders related to PAP gene, such as described in the specification. Said decrease in activity may relate for example to reduced level of expressed PAP, for example if the promoter region of the PAP gene has been altered, or to disrupted structure or function of PAP enzyme. “Decrease in the activity of (expressed) PAP” refers not only to expressed PAP which has lower activity compared to normal PAP, but also to PAP which is expressed at lower levels than normally. This embraces also PAP which is substantially not expressed at all.

The “PAP gene” as used herein refers to any suitable (transmembrane) prostatic acid phosphatase gene or homologue or derivative thereof. The PAP gene to be used in the present invention may be of any suitable origin, plant or animal, such as rat, mouse or human PAP gene. In one embodiment the human prostatic acid phosphatase (hPAP) gene is used to provide a model for investigating human PAP-related disorders. The human PAP gene may be inserted to another species, such as rat or mouse, or to a cell thereof, to provide a transgenic animal or cell model for investigating said disorders or diseases. Also PAP genes of other origins may be used, such as mouse PAP (mPAP) or rat PAP (rPAP). Generally any PAP gene from any suitable organism, which will produce, when expressed in a cell, a substantially functional PAP enzyme, may be used. Such PAP gene may be a homologue of a known PAP gene from certain species having insertions or deletions of amino acids, but still having sufficient homology with the original gene to produce substantially analogous PAP enzyme. Generally such homology is preferably at least 50%, more preferably 80% and most preferably 90% at amino acid level. The “fragment” of TM-PAP refers to a C-terminal fragment having the transmembrane part.

“Vesicle transport disorder” as used herein refers to a disorder wherein the function of the transport mechanism of the cell is disrupted (trafficking disease). This can also be called an exocytosis and/or endocytosis disorder which may lead to several diseases, such as cancer. In this mechanism involved are so called SNARE complexes. The primary role of SNARE proteins is to mediate fusion of cellular transport vesicles with the cell membrane at the porosome or with a target compartment (such as a lysosome). SNARE interacts with the Snapin protein which is regulated by TM-PAP as described herein. The Snapin sequences disclosed in FIGS. 6A-C interact with TM-PAP and they may be the target of the therapy of vesicular trafficking disorders. A molecule capable of binding to any of said sequences may be used as a therapeutic agent for treating said disorders. One example of such a molecule is TM-PAP as described herein.

“Phosphatidylinositol phosphate” (PIP) refers to any phosphatidylinositol phosphate as described herein, such as phosphatidylinositol 3-phosphate PI(3)P, phosphatidylinositol 4-phosphate PI(4)P, PI(5)P, PI(3,5).sub.2, PI(4,5)P.sub.2, PI(3,4,5)P.sub.3 or corresponding soluble inositol phosphate (IP).

The present invention also utilizes knockout/knockdown animals and cells wherein at least one allele of an endogenous transmembrane prostatic acid phosphatase (TM-PAP) gene is functionally disrupted in somatic and/or germ cells. Said animals or cells may be heterozygous or, preferably, homozygous for the TM-PAP knockout/knockdown.

Animals to be used in the animal model of the present invention include any suitable non-human animals, such as vertebrates, or more particularly mammals. The term animal includes an individual animal in all stages of development, including embryonic and fetal stages. In one embodiment of the invention the animal is a rodent, such as mouse or rat, which are generally used as similar applications may be adapted to both species. The cells to be used include any suitable cells, from plants or animals, for example ones derived from the animal described above or human cell lines.

The disruption may be made to exon 3 of the TM-PAP gene. This is preferred because exon 3 is involved with TM-PAP activity. This will ensure that the activity of the TM-PAP enzyme will be abolished. Said exon 3 may be knocked out totally or its function may be decreased partially. However, similar disruptions or knockouts may be also made to another suitable part of TM-PAP gene, for example by introducing deletions or insertions of nucleic acids to obtain the defective TM-PAP. The TM-PAP gene may also be totally removed. Said disruption may also refer to the level of expressed gene product.

Said disruption may be introduced into said transmembrane prostatic acid phosphatase gene by replacing at least part of it with an external nucleic acid sequence. One example of such external nucleic acid sequence is the commonly used neo cassette.

Said transmembrane prostatic acid phosphatase gene may be originated from different species, such as human, mouse or rat prostatic acid phosphatase gene or homologue thereof, or it is a recombinant TM-PAP gene.

Said external nucleic acid molecule used for replacing part of the TM-PAP gene may be any suitable nucleic acid molecule containing suitable nucleic acid sequence which is able to decrease the activity or level of expressed prostatic acid phosphatase when inserted to the TM-PAP gene, for example at the location of exon 3. Targeting to generate a null or mutated allele is usually accomplished by insertion of a selectable marker into a gene causing disruption of splicing, promoter function, or reading frame, with or without deletion of some of the gene. One commonly used selectable marker gene for making knockouts is the neo gene, which confers resistance to the antibiotic neomycin. Also other suitable sequences may be used.

One embodiment of the present invention provides methods for testing and screening a compound, such as a drug candidate, for a therapeutic effect, comprising: administering said compound to a cell or a non-human animal having disruption in the prostatic acid phosphatase (PAP) gene or regulation thereof resulting in a decrease or absence of the activity or the level of prostatic acid phosphatase, and determining if said compound substantially restores the unbalanced phosphatidylinositol phosphate cascade and/or signaling pathway related to PAP expression or activity on said cell or said animal, said restoring indicating said compound being therapeutically effective for treating vesicle transport disorders or disorders related to unbalanced phosphatidylinositol phosphate cascade and/or signaling pathway.

In one embodiment of the present invention said restoring is decrease in PI(4,5)P.sub.2 accumulation. In another embodiment of the present invention said restoring is decrease in the level of PI(3)P.

In one embodiment of the present invention the response on a similar animal or cell not exposed to said compound is determined and the responses are compared in order to find out if there is an effect for said compound.

Such control animal may be exposed for example to placebo or other compound. In one embodiment the response of a knockout/knockdown animal or cell is compared to one of a wild type animal. In other embodiments, the knockout/knockdown animals or cells are examined directly without comparison to a wild-type animal. Such methods are generally well known in the art. Said therapeutic effect is against a disorder related to transmembrane prostatic acid phosphatase, such as any vesicle transport disorder. Non-limiting examples of such diseases or disorders are prostatic atypical hyperplasia, prostatic intraephitelial neoplasia, carcinoma; prostatitis, respiratory or kidney diseases; or inflammatory, immunodefence or autoimmunediseases and other disorders related to phosphorylation/dephosphorylation or transport of phosphatidylinositol 3-phosphate (PI(3)P). As TM-PAP is now known to reduce the amount of PI(3)P, said disorder may be associated with the PI(3)P metabolism and related mechanisms, such as insulin response, lipid metabolism, growth factor response, cell division, apoptosis etc., which are regulated by PI(3)P. Said diseases and disorders include prostate, bladder and pancreatic cancer, myopathy (myodegeneration) and neuropathy (neurodegeneration). A compound found with the method of the invention may be used as medicament for treating the disorders disclosed herein. Therefore, one embodiment of the present invention provides a therapeutic compound obtained with the method on any of the preceding claims. Non-limiting example of such compound may be a compound acting as an enhancer for the expression of TM-PAP.

One embodiment of the present invention provides methods for treating said diseases or disorders related to TM-PAP by gene therapy and other methods to restore the activity of TM-PAP. A compound affecting to the expression or effect of TM-PAP may be administered to tissues or cells of a patient suffering a condition or disorder related to TM-PAP, as described above. Also gene therapy affecting to or restoring the activity of TM-PAP may be given to such patient. The gene therapy preferably restores at least part of the TM-PAP activity. Suitable genes to be used in such gene therapy include any suitable TM-PAP gene or nucleotide vector of the invention as described herein. The gene therapy or other treatment may be administered to the patient for example by injection to target, such as to prostate or by prostate artery, as a virus vector. Such gene therapy methods are generally well known in the art and a person skilled in the art can choose a suitable method for the treatment. Examples of commonly used virus vectors which may be used to carry the TM-PAP gene include baculo, adeno and lenti viruses (see e.g. Airenne et al.: “Baculovirus-mediated Gene Transfer: An Evolving New Concept”, Templeton N S, ed. Gene and Cell Therapy, 181-197, New York, Marcel Dekker, 2004; Kost et al.: “Baculovirus as versatile vectors for protein expression in insect and mammalian cells, Nature Biotechnology 2005:23, 567-575). In one example the method for increasing the level or restoring the activity transmembrane prostatic acid phosphatase protein comprises giving to the patient suffering from said disorder gene therapy by administering a gene encoding the transmembrane prostatic acid phosphatase protein or the C-terminal part thereof.

To find out the physiological function of PAP in the prostate PAP knockout (KO) mouse model was generated by deleting the 3.sup.rd exon of the mPAP gene. The present inventor found out that PAP is PI(3)P-lipid phosphatase, regulates PI(3)P membrane traffic, and its inactivation is causative for prostate cancer. The corresponding publication (Vihko P T, Quintero I, Ronka A E, Herrala A, Jantti P, Porvari K, Lindqvist Y, Kaija H, Pulkka, A, Vuoristo J, et al. 2005 Prostatic acid phosphatase (PAcP) is PI(3)P-phosphatase and its inactivation leads to change of cell polarity and invasive prostate cancer. p1328 (#5239) Proceedings of the AACR: 96th Annual meeting, Anaheim, Calif.) is incorporated herein by reference. Materials, methods, experiments and discussion for preparing a PAP knockout mouse and testing thereof are described in WO06051172A1. As the TM-PAP is encoded by the same gene as PAP, said PAP animal model can be directly applied to TM-PAP the applications thereof.

Prostatic Acid Phosphatase is not a Prostate Specific Target (Quintero et al., Cancer Res 2007; 67: (14) 6549-6554).

Experimental Procedures Antibodies

Lysosomal associated membrane protein (LAMP) 2 monoclonal antibody was developed by J. T. August and J. E. K. Hildreth (Johns Hopkins University) and obtained from the Developmental Studies Hybridoma Bank (developed under the auspices of the NICHD and maintained by The University of Iowa, Department of Biological Sciences, Iowa City, Iowa 52242). Mouse monoclonal antibody against bis(monoacylglycero)phosphate (BMP) was a generous gift from Prof. Jean Gruen berg (University of Geneva). PAP polyclonal antibody was generated by the present inventor's group. Flotillin-1 was from BD Biosciences.

PCR-Based Cloning of Novel PAP Variants

To obtain cDNA encoding rat TM-PAP, total RNA isolated from rat prostate was amplified by RT-PCR using primers 5′-ACCATGAGAGCTGTCCCTCTG-3′ and 5′-TCAGATGTTCCGATACAC-3′ designed from the rat genomic region which was found in silico based on inventor's hypothesis of high similarity in rat and mouse PAP gene structure and the peptide sequence of a possible TM domain.

To obtain a cDNA encoding human TM-PAP, RT-PCR was performed using total RNA from human prostate as a template and primers 5′-ATGAGAGCTGCACCC-3′ and 5′-GCTCTGGGCAGATTCAAAAGG-3′.

The resulting RT-PCR products were cloned into a pCRll-TOPO vector (Invitrogen) and sequenced.

Splice Variant-Specific RT-PCR

Reverse transcription of total RNA to cDNA and subsequent amplification were performed using the GeneAmp RNA PCR kit (Applied Biosystems) according to the manufacturer's instructions. Total RNA was isolated from human and mouse tissue specimens, mouse fibroblasts, SW10, LNCaP and PC-3 cells, and used as a template. Primers are described in supplementary data.

Quantitative real-time RT-PCR to amplify cDNA fragments for the individual PAP variants in human prostate tissue specimens was performed by using TaqMan chemistry on an ABI Prism 7700 sequence detection system (Applied Biosystems). Human tissue specimens were obtained as redundant material for diagnosis from hormonally untreated patients going through transurethral resection for benign prostatic hyperplasia (BPH) or total prostatectomy in the case of prostate cancer (PC) in the Oulu University Hospital. Histopathology was confirmed and cancer tissue dissected by the pathologist. The cDNA was synthesized from 0.5.mu.g total RNA derived from frozen prostate tissue samples, BHP (n=27) and PC (n=19). Primers are described in supplementary data. TM-PAP mRNA variant, PAP mRNA secreted variant, and 18S RNA levels were measured. The results were normalized to 18S quantified from the same samples. Statistical analyses were performed with Student's two-tailed t-test. Difference was considered significant if P<0.05.

The permissions to use the human (prostate and muscle) and mice specimens for research purposes have been obtained from Oulu University Hospital's Ethical Committee and National Authority for Medicolegal Affairs, and from The Animal Experimentation Committee, University of Oulu, Finland, respectively.

Cell Culture

The human prostate carcinoma cell lines PC-3 and LNCaP, and mouse neuronal Schwann cells (SW-10) were obtained from American Type Culture Collection (ATCC) and grown according to the instructions of ATCC.

Mouse skin fibroblasts were obtained by culturing of two-day-old mouse skin explants and subcultured.

Immunofluorescence and Confocal Microscopy

Immunolabeling of human samples was performed according to Andrejewski et al. (Andrejewski N, Punnonen E L, Guhde G, et al. Normal lysosomal morphology and function in LAMP-1-deficient mice. J Biol Chem 1999; 274: 12692-701). Samples were fixed in 4% paraformaldehide/2.5% sucrose, mounted on sample holders and frozen in liquid nitrogen. Cryosections for fluorescence and electron microscopy were cut at −100.degree. C. Fluorescent (Alexa Fluor 594 goat anti-mouse IgG and Alexa Fluor 488 goat anti-rabbit IgG, Molecular Probes) or gold-conjugated (5-nm gold goat anti-mouse IgG and 10-nm goat anti-rabbit IgG, British Bio Cell, Gardiff) secondary antibodies were used. The samples were analyzed using a confocal microscope (Leica Microscopy and Scientific Instruments Group) or a Jeol 1200 EX electron microscope (Tokyo).

LNCaP and SW-10 cells grown on Thermanox plastic coverslips (Nalgene-Nunc, Rochester, N.Y.) were subjected to pre-embedding immunolabelling with anti-PAP antibody (Vihko P, Sajanti E, Janne O, Peltonen L, Vihko R. Serum prostate-specific acid phosphatase: development and validation of a specific radioimmunoassay. Clin Chem 1978; 24: 1915-9) as described earlier (Uchiyama K, Jokitalo E, Kano F, et al. VCIP135, a novel essential factor for p97/p47-mediated membrane fusion, is required for Golgi and ER assembly in vivo. J Cell Biol 2002; 159: 855-66) and processed for epon embedding. Sections were cut parallel to the coverslip, post-stained with uranyl acetate and lead citrate, and imaged with Tecnai 12 (FEI Corp.) electron microscope at 80 kV.

LNCaP and PC-3 cells grown on cover slips were fixed with 4% paraformaldehyde, and permeabilized with blocking buffer (1% BSA/0.2% saponin in PBS). Primary antibodies were incubated for 1 hour at room temperature. Fluorescent second antibodies were used (Alexa Fluor 488 goat anti-rabbit IgG (Molecular Probes) and TRITC goat anti-mouse IgG (Sigma)), and 4,6-diamidino-2-phenylindole (DAPI, Fluka chemicals) to stain the nucleus. Cells were embedded in Immuno Mount (Thermo Sandon). Confocal imaging was performed using Olympus FluoView 1000 confocal microscope.

Transfection of PC-3 Cells with TM-PAP-GFP

cDNA for human TM-PAP was obtained by RT-PCR using human prostate total RNA as a template and primers as follows: 5′-TTAGGATCCACCATGAGAGCTGCACC-3′ and 5′-AATGGATCCGATGTTCCCATAGGATTC-3′. The PCR product was cloned into pCR 2.1-TOPO plasmid (Invitrogen). From the recombinant plasmid, the BamHI restriction fragment containing the coding region of TM-PAP was cloned into a pEGF-N3 (BD Biosciences Clontech) plasmid. Restriction digestions and sequencing confirmed the direction of the insert and sequence of the construction. PC-3 cells were grown to 80-90% confluence and transfected using Lipofectamine 2000 (Invitrogen) according to manufacturer. Transfection (DNA:Lipofectamine, 1:2) was performed for 5 hours at 37.degree. C. in Opti-MEM medium (Gibco). Cells were incubated in normal growth medium for 24 hours at 37.degree. C. before experiments.

Immunohistochemistry

Histopathologically normal skeletal muscle biopsies were chosen for the study (from the files of the pathology laboratory, Oulu University Hospital, Finland). The muscle biopsies were orientated and mounted in optimum cutting temperature (OCT) compound, frozen in isopenthane and liquid nitrogen, and cut for immunohistochemistry (10.mu.m). The immunohistochemistry procedure was performed using an anti-PAP antibody (1:500) (Vihko P, Sajanti E, Janne O, Peltonen L, Vihko R. Serum prostate-specific acid phosphatase: development and validation of a specific radioimmunoassay. Clin Chem 1978; 24: 1915-9), the EnVision (DAKO) staining kit and diaminobenzidine as chromogen (DAKO).

Disturbed Interaction of Transmembrane Prostatic Acid Phosphatase and Snapin is Associated with Prostate Adenocarcinoma

To explore the underline mechanisms and understand the physiological function of PAP, the prostate of the PAP-deficient mouse model (PAP^{-/-}), and a stable transmembrane PAP-transfected LNCaP cell line were studied. The PAP^{-/-} mouse prostate showed the development of slow-growing non-metastatic prostate adenocarcinoma, with similar features to the human counterpart. Disturbed vesicular traffic and changes in the cell polarity were observed by electron microscopy in the PAP^{-/-} mouse DLP, and the differential gene expression analyses of PAP^{-/-} mouse prostates revealed deregulation of genes related to vesicular traffic, confirming the ultrastructural findings. To discover the interacting partners of PAP, yeast two-hybrid assays were carried out and a clear result was obtained by the interaction of PAP with Snapin, a SNARE-associated protein involved in the membrane fusion process. This interaction was confirmed by co-localization. The results suggest that disturbed vesicular traffic is a hallmark of prostate adenocarcinoma.

Topologically, TMPAP protein resides in the plasma membrane and in the endosomal-lysosomal pathway, including multivesicular endosomes. The N-terminal phosphatase activity domain is extracellular when TMPAP is in the plasma membrane and intra-luminal when it is trafficking in vesicles. Its C-terminal domain contains a cytosolic tyrosine-based endosomal-lysosomal targeting signal motif (YxxΦ). The current evidence does not support the existence of a third, cytosolic cellular form of PAP, as it has been suggested in the literature but never cloned.

SNARE proteins comprise a large family found in yeast and mammalian cells, with the primary function to mediate docking and fusion of vesicles with the cell membranes. Snapin is a SNARE-associated protein interacting with Snap25 and Snap23 proteins and increasing the binding of synaptotagmins to the SNARE complex. Snapin also forms part of the BLOC1 protein complex, which is necessary for the biogenesis of vesicles in the endosomal-lysosomal pathway.

To understand the physiological function of PAP, the prostate of the PAP-deficient mouse model (PAP^{-/-}) was studied (Vihko, 2005: Proceedings of the AACR Abstract 5239: 96th Annual Meeting. Anaheim, Calif., USA). The mouse prostate consists of three different lobes: anterior (AP), dorsolateral (DLP) and ventral prostate (VP); and it does not show spontaneous development of neoplasia in mice.

The PAP^{-/-} mouse prostate showed disturbed vesicular trafficking, changes in cell polarity and development of slow-growing non-metastatic prostate adenocarcinoma. Herein the interaction of TMPAP with snapin is reported, and it is suggested that disturbed exocytosis is a hallmark of prostate adenocarcinoma.

Materials and Methods

Mice.

Mice deficient in PAP were generated by replacing exon 3 (ACPPΔ3/Δ3) of the prostatic acid phosphatase gene (ACPP, PAP) with the neo gene as described earlier (Vihko, 2005) thereby abolishing the expression of both PAP isoforms. The fertility status in the PAP^{-/-} mice was not affected by the gene modification. PAP^{-/-} mice were backcrossed to the C57BL/6J strain (Harlan Laboratories Inc.) for 16 generations to obtain homogenous background. Age-matched C57BL/6J male mice were used as controls in all the experiments. The animal protocols were approved by the Animal Experimentation Committee of the University of Oulu and ELLA—The National Animal Experiment Board of Finland. The project license numbers are 044/11 and STH705A/ESLH-2009-08353/Ym-2.

Histology.

Mouse prostates were formalin-fixed and embedded in paraffin. Five μm serial sections were stained with hematoxylin and eosin and evaluation of histology was performed following the previously described guidelines. Six to eight mice of each age group were analyzed

Smooth muscle actin and pan cytokeratin staining.

Five μm sections of paraffin-embedded mouse prostates were deparaffinized, rehydrated and treated for antigen retrieval in microwave oven 10 min in 0.01 M sodium citrate buffer, pH 6. The smooth muscle actin was detected with mouse monoclonal anti-smooth muscle β-actin antibody (ASM-1, 1:200, Progen Biotechnik), and the epithelial cells were stained with mouse monoclonal anti-pan cytokeratin (1:250, Abcam). Unspecific binding of the antibody was blocked using BEAT™ Blocker Kit (HistoMouse™, Life Technologies). Immunohistochemical detection was performed with Histomouse Max DAB-Kit (Life Technologies). Specimens were counterstained with hematoxylin. Images were taken with Labovert FS microscope (Leica Microsystems GmbH).

Proliferation and Apoptosis Analyses.

The cell proliferation and apoptosis indexes were determined for the PAP^{-/-} and PAP^{+/+} DLP lobes from 3, 6 and 12 month old mice, 4 mice per group. Ki67 staining (Abcam) with Vectastain Elite ABC Kit (Vector Laboratories) was used to detect the proliferating cells, which counterstained with hematoxylin. Terminal deoxynucleotidyl transferase-mediated dUTP nick end labeling (TUNEL) staining of the apoptotic cells was conducted with FragEL DNA Fragmentation Detection Kit (Calbiochem) following the manufacturer's instructions. Ki-67- and TUNEL-positive cells were quantified as follows: 5 μm consecutive sections of paraffin-embedded DLP lobes were either Ki-67 or TUNEL stained. Sections were photographed under Labovert FS microscope (magnification 40×), 10 random fields from each section. Each field was counted for proliferative and apoptotic status. The ratio of positive cells to the total amount of cells per sample were compared between PAP^{+/+} and PAP^{-/-} groups using two-sample t-test for equality of proportions with continuity correction for each age group as implemented in R statistical package version 2.10.1.

Transmission Electron Microscopy.

DLP samples from age-matched PAP^{-/-} and PAP^{+/+} mice and human prostate specimens were fixed in a mixture of 1% glutaraldehyde and 4% formaldehyde in 0.1 M phosphate buffer for TEM. The samples were post-fixed in 1% osmium tetroxide, dehydrated in acetone, embedded in Epon Embed 812 (Electron Microscopy Sciences) and analyzed at the Biocenter Oulu EM core facility using Philips 100 CM Transmission Electron Microscope with CCD camera.

Human samples were obtained during total prostatectomy of T1/T2 (TNM Classification of Malignant Tumors) prostate cancer.

Yeast Two-Hybrid Analysis.

To screen for interacting partners of human TMPAP, yeast two-hybrid screening was performed using the Matchmaker Gal4 two-hybrid System 3 (Clontech) in accordance with the manufacturer's instructions. The bait construct consisted of the coding region of human TMPAP (GeneBank accession BC007460, nucleotides 51-1304, except the starting methionine was changed to valine) cloned in frame into NcoI/SmaI sites of pGBKT7 using PCR generated linkers. A human thymus cDNA library cloned in pACT2 (Clontech) was used as the prey. The bait and prey plasmids were co-transformed into Saccharomyces cerevisiae May 203 strain according to the Clontech's two-hybrid protocols. Inserts of positive clones were amplified by PCR, and the DNA was automatically sequenced.

Generation of stable transfected LNCaP cells.

TMPAP production in LNCaP cells: Human TMPAP cDNA was cloned between BamHI-NotI in pMX neo vector. The human Phoenix gag-pol packaging cell line was cultured in high-glucose DMEM containing 10% FCS, 2 mM glutamine, and 100 unit/ml penicillin and 100 μg/ml streptomycin (Sigma). Confluent cells in six-well plates were transfected with 4 μg of pMX neo-TMPAP or pMX neo and 0.4 μg of pVSV-G retroviral vector per well by using Lipofectamine 2000 (Invitrogen) according to the manufacturer's instructions. Twenty-four hours post-transfection, the medium was changed to low-glucose DMEM with the same supplements as above. Forty-eight hours post-transfection, the viral media was collected every 24 h for up to 6 days. LNCaP cells (ATCC) were cultured in RPMI with 10% FCS, 2 mM glutamine, and 100 U/ml penicillin and 100 μg/ml streptomycin (Sigma) in six-well plates. When cells were 70% confluent the medium was changed to 1 ml of virus-containing supernatant with 4 μg/ml polybrene (Sigma) per well and incubated at 37° C. Twenty-four hours after the first infection, the medium was removed and cells were infected again as above. Forty-eight hours after the first infection, cells were trypsinized and seeded on a six-well plate in presence of 400 ug/ml geneticin (Roche) for selection. Antibiotic selection was continued until cells on the control wells have died. TMPAP production was assayed by quantitative RT-PCR or immunoblotting.

Immunofluorescence and Co-Localization Studies.

LNCaP pMX TM-PAP and LNCaP pMX cells were seeded on 12-mm glass coverslips (BD Biosciences) and fixed with 4% paraformaldehyde followed by 5 min methanol at −20° C., and blocked using 0.2% Saponin, 5% fat free dry milk in PBS for 30 min. Double immunostaining: cells were incubated overnight at 4° C. with primary mouse monoclonal anti-human PAP (1:100, Sigma) and rabbit polyclonal anti-snapin (1:200, Synaptic System) antibodies. Cells were then incubated with Alexa Fluor 488 goat anti-mouse, and Alexa Fluor 594 goat anti-rabbit (Invitrogen) antibodies for one hour at RT. The samples were mounted (Mowiol/DABCO/DAPI), and confocal images were acquired using an Olympus FluoView 1000 confocal microscope with UPLSAPO 60× oil NA 1.35 objective, and 405, 488, and 543 nm laser excitation. Three different experiments were performed and three different fields were analyzed per sample. Seven to thirteen stack images were acquired for each field using sequential scanning, with an image size of 1024×1024 and 500 nm in z-dimension.

Confocal stack images were deconvolved with 5 iterations using AutoQuantX's blind deconvolution algorithm (Media Cybernetics). The 3D co-localization studies were performed using Biplane Imaris 7.2 software (Biplane AG) and the automatic threshold co-localization algorithms developed by Costes et. al, (Biophysical Journal, 86: 3993-4003, 2004). The co-localization results are expressed as a mean of Pearson's coefficient±SEM.

Statistical Analyses.

The proliferation and apoptosis studies were analyzed using the two-sample t-test for equality of proportions with continuity correction as implemented in R statistical package version 2.10.1. FDR-corrected P-values were calculated in microarray experiments. For other experiments we used Student's two-tailed t-test for statistical analyses. Differences between groups were considered significant when P-value <0.05. In co-localization analyses, Pearson's correlation coefficient was calculated and expressed as mean±SEM.

Accession Codes.

Gene expression files containing microarray raw-data can be accessed from ArrayExpress repository database (accession number E-MTAB-1191).

Results

Effect of PAP Depletion in Mouse Prostate.

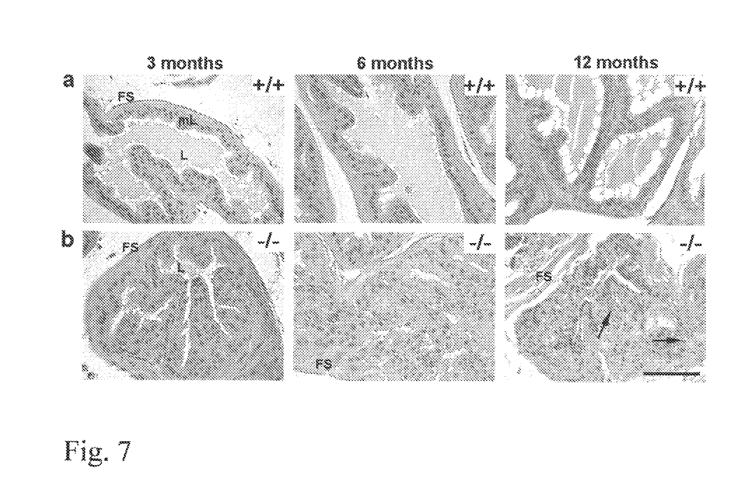

The removal of PAP led to slow development of prostate neoplasia in DLP and AP. Hyperplastic growth, tufting, and micropapillary structures of the epithelial cells were observed at the age of 3 months, followed by mouse prostatic intraepithelial neoplasia (mPIN) development at the age of 6 months, and prostate adenocarcinoma at the age of 12 months (FIG. 7). The follow-up of the disease in mice spanned until 26-month-old, where the presence of other pathologies arose as strain background or due to mouse aging.

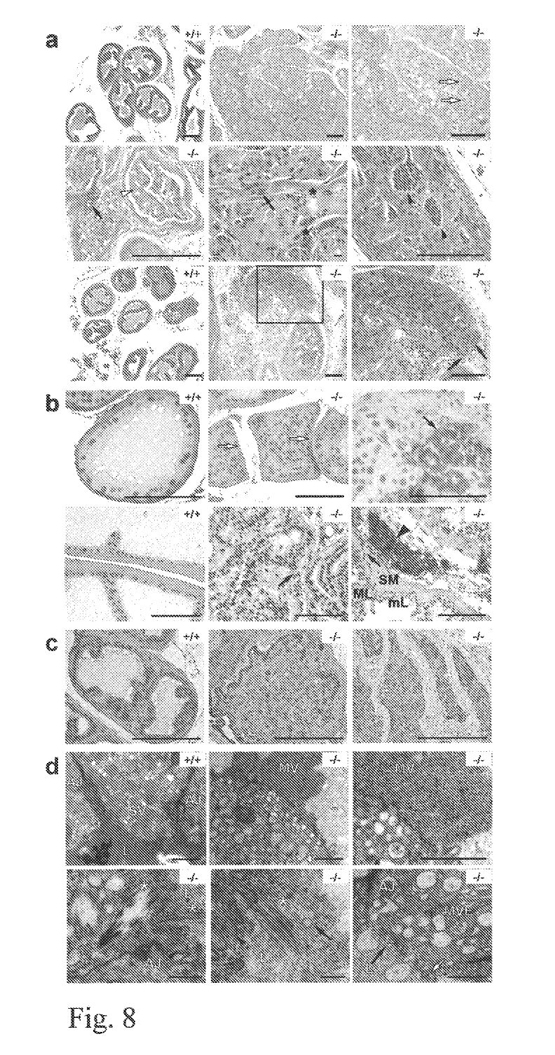

All the one-year-old PAP^{-/-} mice developed prostate adenocarcinoma. Similarly to humans, normal and cancerous acini were present in mouse prostate. Pathological acini were filled with epithelial cells containing enlarged nuclei which showed some variation in their sizes. The nuclei often displayed prominent nucleoli and the cells exhibited a loss of cohesion. In addition, the acini were surrounded by a fibrotic stroma to which some of the cell clusters appeared to be invading with bulging areas and fusion. In other areas, the epithelial cells lining the acini formed papillary fronds, and blood vessels were present among neoplastic epithelial cells. The histological pattern was consistent with locally invasive prostate adenocarcinoma (FIG. 8a). However, no metastatic lesions were detected in other studied organs (brain, liver, lungs and lymph nodes).

The breakdown of the fibromuscular sheath and invasion of the epithelial cells into stroma was also detected with smooth muscle β-actin (SMA) staining. Bulging of the cells could be seen with decreased intensity of SMA in atypical acini, as well as adenocarcinoma invasion. Crowding of inflammatory cells was detected in sites of microinvasive adenocarcinoma (FIG. 8b). The presence of neoplastic acinar cells in the lumen was confirmed with pan cytokeratin staining (FIG. 8c).

Proliferation and apoptosis in PAP^{-/-} mouse DLP.

Due to the gradual increase in prostate cell amount, we determined the status of proliferation and apoptosis in the tissue. As a result, the proliferation was significantly increased at three—(P-value=4.3×10-3, n=4), six—(P-value=1.3×10-15, n=4) and 12-month-old (P-value=3.9×10-5, n=4) PAP^{-/-} mice DLP, but the apoptosis status was not different between genotypes (P-values 0.3, 0.1, and 0.9 respectively, n=4).

Ultrastructural Changes in PAP^{-/-} Mouse DLP.

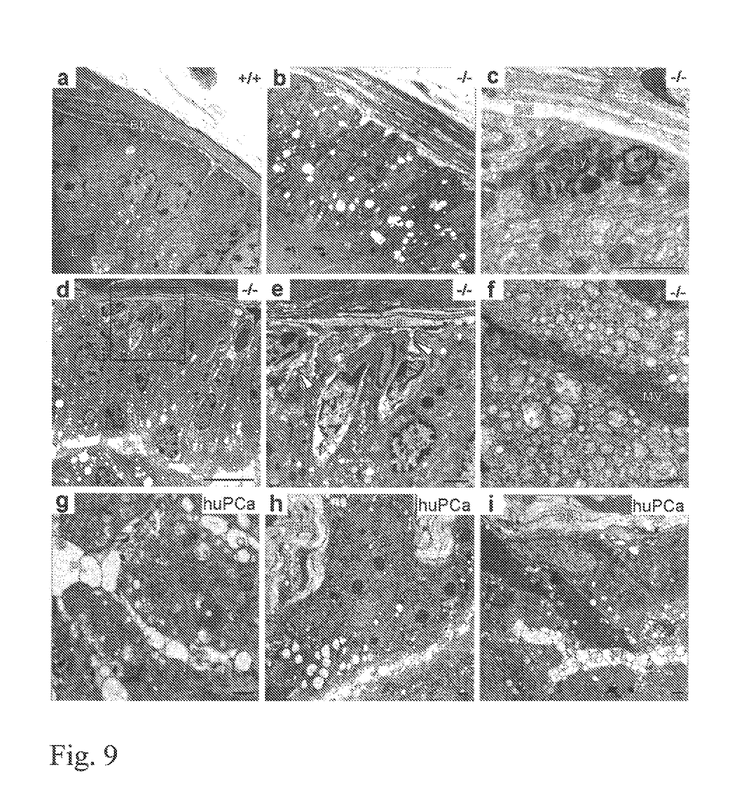

Further analysis of prostate ultrastructural changes by transmission electron microscopy (TEM) showed an increased number of electron-lucent enlarged vacuoles and luminal exosome-like vesicles. Lamellar body-like structures were observed in PAP^{-/-} mice DLP cells and their contents were secreted into the lumen. In addition, the lumen of the acini was often filled with membranous material that consisted of microvesicles, 50-100 nm, as well as other membranous structures (FIG. 8d). Other important changes observed included irregularities and invaginations of the basement membrane into the epithelium, in addition to the presence of lysosomes in the basal side of the cell, microvesicles in the intercellular space and disintegration of the apical microvilli, all indicating loss of cell polarity. The prostatic epithelium of PAP^{-/-} mice lost the regular structure of uniform monolayer and became a multilayer epithelium with presence of pseudo-lumens. Similarly, human prostate cancer (huPCa) TEM pictures showed high number of electron-lucent enlarged vacuoles in the epithelial cells, as well as accumulation of membranous material in the lumen of the acini, and irregular basement membrane (FIG. 9).

Differential Gene Expression in PAP^{-/-} Mouse DLP.

To determine the effects of PAP deficiency at gene expression level, we performed microarray experiments of mouse prostatic tissue. The Gene Ontology analyses of differentially expressed genes showed, among the most significant groups, those genes associated with regulated secretory pathway, neurotransmitter secretion (Snap25, Syt1, Syt4, Nrxn1, Cart, Abpa, Cplx1) and synaptic vesicle traffic (Rab3c, Syp, Syn2, Syngr1, Syt1, Syt4, Syt10, Svop, Bcan, Timp4). Comparative genomic hybridization analysis of DNA prostate samples performed at 6-, 15- and 25-month-old showed no differences in copy number between the genotypes (wild type C57BL/6J and PAP^{-/-} mice, data not shown).

Yeast Two-Hybrid Screening.

The disturbed exocytosis observed in the ultrastructural studies of PAP^{-/-} prostates and the differential expression of genes related to vesicle fusion, such as Snap25, Syt1, Syt4 and Cplx1, led us to search for proteins interacting with TMPAP. The yeast two-hybridization screening of human thymus library detected seven out of 15 clones expressing snapin (NM_{—}012437.3), as a clear candidate for interaction with TMPAP.

Determination of PAP/Snapin Interaction.

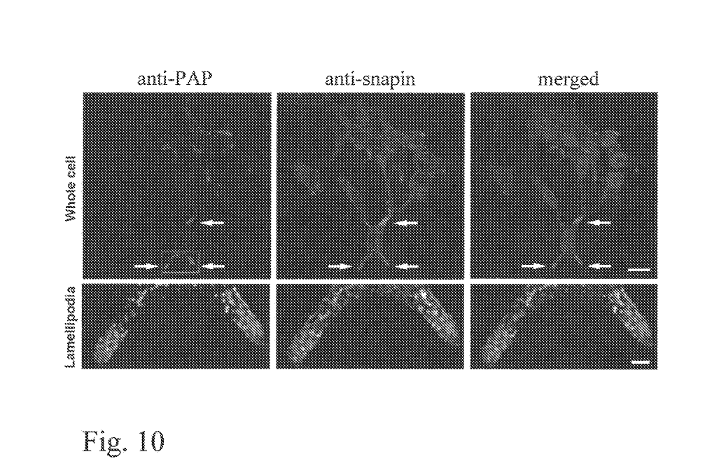

To validate this finding, double-immunofluorescence staining of PAP and Snapin in TMPAP stable transfected LNCaP cells showed co-localization of these two proteins in vesicular structures and cell membrane (FIG. 10a). The quantification studies displayed relatively low Pearson's correlation coefficient when the whole cell was analyzed (0.485±0.012). However, when the co-localization was quantified exclusively in the cell lamellipodia the Pearson's correlation coefficient reached a value of 0.680±0.013. This coefficient value could imply an interaction between TMPAP and Snapin in these cell regions.

There is a clear lack of information related to suitable partners for PAP, perhaps due to the fact that for many years just the secretory form has been studied. The description of the second isoform of PAP has open new views about the function of this protein. Both neurons and prostate epithelial cells are polarized cells.

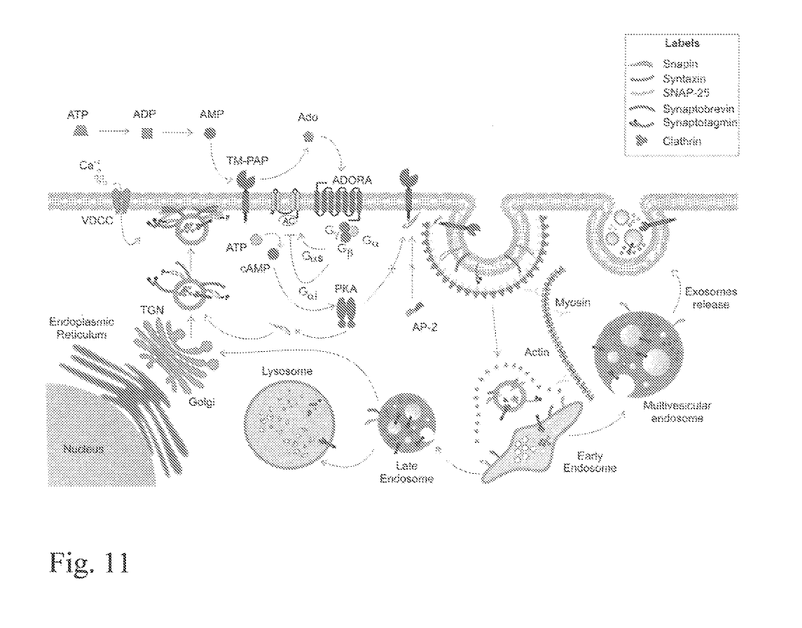

Considering the topology of TMPAP, this enzyme cannot exert cytosolic acid phosphatase activity and consequently it is not able to dephosphorylate cytosolic tyrosines of epidermal growth factor receptor (EGFR) as it has been previously suggested. Therefore, it is assumed that this is not the pathway leading to the observed prostate adenocarcinoma in the PAP^{-/-} mice. In FIG. 11, the hypothesis about the modulatory effect of TMPAP on exocytosis and the mechanisms involved in the physical interaction between TMPAP and snapin is summarized. Previous reports showed that the interaction of membrane proteins with snapin negatively regulates exocytosis by affecting the coupling of synaptotagmin to the SNARE complex or by reducing Snapin phosphorylation.

The present results are consistent with these previous findings, and based on all the information available a mechanistic model was built representing the interaction between TMPAP and Snapin. First, according to Buxton et al. (Biochem. J. (2003) 375, 433-440) 70% of Snapin is found in the cytosol. Assuming that phosphorylation of Snapin by the cAMP-dependent kinase PKA occurs in cytosol, this process would be delayed if Snapin is bound to TMPAP at the vesicle or plasma membrane. This could be a first regulatory effect on secretion. A second effect could involve the 5′-ectonucleotidase activity of TMPAP responsible for the production of adenosine from AMP. In this case, adenosine could bind to its cognate receptors A1, A2 and A3 that modulate cAMP levels. Considering that PKA activity is regulated by cAMP, the phosphorylation of Snapin would be affected by this process. Moreover, the interaction between TMPAP and Snapin at the plasma membrane could block the interaction between the cytosolic YxxΦ motif in TMPAP and the adaptor protein complex-2 required for clathrin-based endocytosis. This effect could delay the internalization of TMPAP and extend the time that TMPAP is present in the cellular surface eliciting its catalytic activity and producing a sustained adenosine effect on adenosine receptors. According to this model, the lack of TMPAP would lead to the dysregulation of vesicular traffic and exocytosis observed in PAP^{-/-} mouse prostate. This could establish a significant starting point for uncontrolled cell proliferation and the development of prostate adenocarcinoma.

Mice Deficient in Transmembrane Prostatic Acid Phosphatase Display Multiple Hallmarks of Schizophrenia

There are two isoforms of Prostatic acid phosphatase enzyme (PAP, ACPP; EC 3.1.3.2): secretory (sPAP) and transmembrane (TM-PAP) (Quintero et al, 2007), splice variants encoded by the same gene (ACPP). TM-PAP is a type 1 transmembrane protein that is also widely expressed in nonprostatic tissues in both sexes. It has a C-terminally located endosomal/lysosomal targeting signal, and in prostate cancer tissue it resides in multivesicular endosomes and lysosomes. TM-PAP also colocalizes with exosomal markers flotillin and Lamp-1. In the DRG, TM-PAP functions as a 5′-ectonucleotidase and produces adenosine that suppresses pain by activating adenosine A1-receptors. An intraspinal injection of sPAP has efficient and long-lasting effects against pain sensation in healthy animals, as well as pain relieving effects in animals sensitized by nerve injury. PAP^{-/-} mice have increased sensitivity for the development of chronic inflammatory and neuropathic pain. Clathrin-mediated endocytosis and clathrin-dependent membrane and protein trafficking have been hypothesized as core pathophysiological mechanisms in neuropsychiatric disorders. The PAP expression and function in the central nervous system is characterized herein in more detail.

Results:

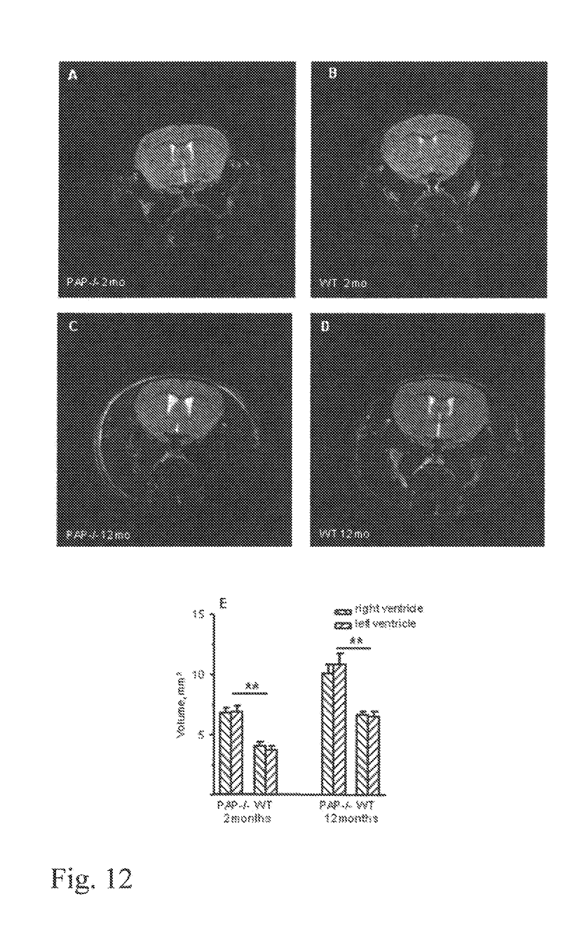

PAP^{-/-} mice had significantly enlarged lateral brain ventricles (FIG. 12); a hallmark of human schizophrenia also seen in several mouse models of the disease. Surprisingly a detailed behavioral analysis showed that PAP^{-/-} mice displayed increased anxiety in the elevated plus-maze test and had a disruption in prepulse inhibition (PPI), a defect in the sensorimotor gating system.

These anatomical, neurochemical, and behavioral characteristics observed in PAP^{-/-} mice suggested that PAP has an important role in the central nervous system and prompted the inventor to characterize its localization and function in the mouse brain. Immunohistochemical stainings showed that TM-PAP is widely expressed in motor-related brain areas, with the most intense PAP-immunoreactivity in cerebellar Purkinje cells, red nucleus, oculomotor nucleus, and in substantia nigra pars reticulata. Also a full-length TM-PAP transcript was cloned from mouse striatal GABAergic neurons (see Materials and Methods), verifying expression of TM-PAP. No sPAP transcript was detected.

The Allen Brain Atlas database (Allen Brain Atlas Resources) was queried for expression of PAP in mouse and human brain. In situ hybridization data of the mouse brain was only available for sPAP (NM_{—}019807), and showed no expression of sPAP in the mouse brain, consistent with the findings. Gene expression data from the developing human brain showed high levels of PAP expression (both sPAP and TM-PAP isoforms) in the ventricular and subventricular zones (Allen Brain Atlas Resources).

Discussion:

On a cellular level, how could TM-PAP regulate GABAergic transmission to produce such a distinct phenotype? TM-PAP has an endosomal/lysosomal targeting signal, and in prostate cancer tissue it is localized in multivesicular endosomes and lysosomes, as well as luminal exosomes (Quintero et al, 2007). In nerve cells, these organelles are used in membrane trafficking pathways controlling recycling and degradation of pre- and post-synaptic membrane proteins, as well as in recycling of vesicle membrane during neurotransmitter release and release of exosomal endocargo. Clathrin-mediated endocytosis has been suggested as a core pathophysiological mechanism in neuropsychiatric disorders. The colocalization of TM-PAP with the presynaptic marker synaptophysin is concordant with previous observations from DRG neurons, where TM-PAP has also been shown to localize presynaptically. TM-PAP also colocalizes with Snapin, which directly binds SNAP-25, a protein that has been linked to schizophrenia in genetic, pathological and functional studies. Snapin is associated with the SNARE complex and involved in synaptic vesicle docking and fusion, supporting the hypothesis that TM-PAP may regulate GABAergic signaling via synaptic vesicle trafficking. The mislocalization of Snapin observed in the cells of PAP^{-/-} mice may perturb synaptic processes controlling neurotransmitter release and recycling, thus disrupting neuronal homeostasis and eventually leading to the schizophrenia-like phenotype observed in PAP^{-/-} mice.

The precise mechanisms involving the pathogenesis of schizophrenia still remain unknown, but it is now viewed as a biochemical condition to whose etiology both genetic and environmental factors contribute. To our knowledge, mutations in the gene encoding PAP (ACPP) have not thus far been reported in schizophrenic patients, nor has ACPP been implicated in genetic association studies of schizophrenia. However, the SNAP-25 locus (Chr: 20p12.3-11) has been implicated in a meta-analysis of genome-wide linkage scans of schizophrenia. GABAergic dysfunctions and alterations in inhibitory circuits of the brain have been implicated in several mouse models of schizophrenia and related disorders. The present findings suggest that by influencing GABAergic signaling through vesicle trafficking, TM-PAP is linked to specific endophenotypes of schizophrenia, and may also be involved in the etiology of other neurological and neuropsychiatric disorders.

Materials and Methods:

All procedures and Experiments involving mice were approved by ELLA—The National Animal Experiment Board of Finland. The project license numbers are STH705A/ESLH-2009-08353/Ym-23 and 044/11.

PAP deficient mice. PAP^{-/-} mice were generated by removing exon 3 (PAPΔ3/Δ3) of the prostatic acid phosphatase gene (PAP, Acpp), completely abolishing expression of sPAP and TM-PAP proteins encoded by the Acpp gene, respectively. PAP^{-/-} mice have been backcrossed to C57BL/6J strain (Harlan Laboratories, Inc.) for 16 generations. PAP^{-/-} male mice were analyzed with age-matched C57BL/6J male mice as controls.

Magnetic Resonance Imaging. Mice 12 months old (wild-type (n=5) and PAP^{-/-} (n=5)) and 2 months old (wild-type (n=4) and PAP^{-/-} (n=4)) were anesthetized with isoflurane for the imaging experiment. MRI studies were performed with a 4.7 T scanner (PharmaScan, Bruker BioSpin, Ettlingen, Germany) using a 90-mm shielded gradient capable of producing a maximum gradient amplitude of 300 mT/m with an 80-μs rise time. A linear birdcage radio frequency coil with an inner diameter of 19 mm was used. After shimming and scout images, coronal T2-weighted 2D images encompassing the whole brain were acquired with using the standard Bruker technique of fast spin echo sequence; rapid acquisition with relaxation enhancement (RARE) sequence (TR/TEeff, 3800/80 milliseconds; Rare factor 8, matrix size, 256×256; field of view, 23×23 mm2; 15 slices, slice thickness 0.5 mm). The body temperatures of the animals were maintained by using a MRI-compatible heating pad (Gaymar Industries, Orchard Park, N.Y., USA). All images were processed using the manual tracing tool provided by ParaVision 4.0 (Bruker BioSpin, Ettlingen, Germany). Manually delineated regions of interest for the right and the left lateral ventricle in each slice were summed up and multiplied by slice thickness yielding the right and left lateral ventricle volumes.

Elevated plus maze. Elevated plus maze test (EPM) was used to measure unconditioned anxiety-like behaviour in mice (PAP^{-/-} n=22, WT n=23).

Prepulse inhibition of acoustic startle response. Sensorimotor gating (PPI) was measured in commercially available system (Med Associates, St. Albans, Ga., USA). Haloperidol was dissolved in saline and administered at the dose of 0.2 mg/kg i.p. 20 min before start of experiment. Number of mice used: PAP^{-/-} n=15 (saline)+15 (haloperidol), WT n=15 (saline)+14 (haloperidol).

Statistics. The behavioural data were analysed using a factorial ANOVA design with genotype and treatment as between-subject factors. A repeated measures ANOVA was applied for analysis of activity data. Post-hoc analysis after significant ANOVA was carried out by means of Newman-Keuls test. For other experiments, data was analyzed with either two-tailed t-test or with repeated measures ANOVA.

Cloning of TM-PAP in striatal GABAergic neurons. Total RNA was isolated from Mouse Brain Striatum Neuronal Cells (Lonza, Basel, Switzerland) using TriReagent (Molecular Research Center, Cincinnati, Ohio, USA). RNA was reverse transcribed into cDNA and subsequently amplified using GeneAmp RNA PCR Kit (Life Technologies Ltd, Paisley, UK). The primers used for TM-PAP amplification by RT-PCR were: 5′-AATCTAGACCATGCCAGCCGTTCCT-3′ (forward, SEQ ID NO: 50) and 5′-CTCTCTAGATCAGATTGTTCCGATACAC-3′ (reverse, SEQ ID NO: 51). The PCR conditions were: 95° C. for 1 min and 45 followed by 30 cycles of 95° C. for 15 s, 63.4° C. for 30 s, and 72° C. for 1 min and 12 s, with the final extension of 7 min at 72° C. PCR product was cloned into pCR2.1 TOPO vector (Life Technologies Ltd.), and bidirectionally sequenced.

Yeast two-hybrid analysis. To screen for interacting partners of human TM-PAP, a yeast two-hybrid screening was performed using the Matchmaker Ga14 two-hybrid System 3 (Clontech, Mountain View, Calif., USA). The bait construct consisted of the coding region of human TM-PAP (GeneBank accession BC007460, nucleotides 51-1304, except the starting methionine was changed to valine) cloned in frame into NcoI/SmaI sites of pGBKT7 using PCR generated linkers. A human thymus cDNA library cloned in pACT2 (Clontech) was used as the prey. The bait and prey plasmids were co-transformed into Saccharomyces cerevisiae May 203 strain according to the Clontech's two-hybrid protocols. Inserts of positive clones (7/15) were amplified by PCR, and the DNA was automatically sequenced.

This invention has been described with an emphasis upon some of the preferred embodiments and applications. However, it will be apparent for those skilled in the art that variations in the disclosed embodiments can be prepared and used and that the invention can be practiced otherwise than as specifically described herein within the scope of the following claims.

Sequence CWU 1

11418PRTHomo sapiens 1Met Arg Ala Ala Pro Leu Leu Leu Ala Arg
Ala Ala Ser Leu Ser Leu1 5 10 15Gly Phe Leu Phe Leu Leu Phe Phe Trp Leu Asp
Arg Ser Val Leu Ala 20 25 30Lys Glu Leu Lys Phe Val Thr Leu Val Phe Arg His Gly
Asp Arg Ser 35 40 45Pro Ile Asp Thr Phe Pro Thr Asp Pro Ile Lys Glu Ser Ser Trp
Pro 50 55 60Gln Gly Phe Gly Gln Leu Thr Gln Leu Gly Met Glu Gln His Tyr Glu65
70 75 80Leu Gly Glu Tyr Ile Arg Lys Arg Tyr Arg Lys Phe Leu Asn Glu Ser 85 90
95Tyr Lys His Glu Gln Val Tyr Ile Arg Ser Thr Asp Val Asp Arg Thr 100 105
110Leu Met Ser Ala Met Thr Asn Leu Ala Ala Leu Phe Pro Pro Glu Gly 115 120
125Val Ser Ile Trp Asn Pro Ile Leu Leu Trp Gln Pro Ile Pro Val His 130 135 140Thr
Val Pro Leu Ser Glu Asp Gln Leu Leu Tyr Leu Pro Phe Arg Asn145 150 155 160Cys
Pro Arg Phe Gln Glu Leu Glu Ser Glu Thr Leu Lys Ser Glu Glu 165 170 175Phe Gln
Lys Arg Leu His Pro Tyr Lys Asp Phe Ile Ala Thr Leu Gly 180 185 190Lys Leu Ser
Gly Leu His Gly Gln Asp Leu Phe Gly Ile Trp Ser Lys 195 200 205Val Tyr Asp Pro
Leu Tyr Cys Glu Ser Val His Asn Phe Thr Leu Pro 210 215 220Ser Trp Ala Thr Glu
Asp Thr Met Thr Lys Leu Arg Glu Leu Ser Glu225 230 235 240Leu Ser Leu Leu Ser
Leu Tyr Gly Ile His Lys Gln Lys Glu Lys Ser 245 250 255Arg Leu Gln Gly Gly Val
Leu Val Asn Glu Ile Leu Asn His Met Lys 260 265 270Arg Ala Thr Gln Ile Pro Ser
Tyr Lys Lys Leu Ile Met Tyr Ser Ala 275 280 285His Asp Thr Thr Val Ser Gly Leu
Gln Met Ala Leu Asp Val Tyr Asn 290 295 300Gly Leu Leu Pro Pro Tyr Ala Ser Cys
His Leu Thr Glu Leu Tyr Phe305 310 315 320Glu Lys Gly Glu Tyr Phe Val Glu Met
Tyr Tyr Arg Asn Glu Thr Gln 325 330 335His Glu Pro Tyr Pro Leu Met Leu Pro Gly
Cys Ser Pro Ser Cys Pro 340 345 350Leu Glu Arg Phe Ala Glu Leu Ala Gly Pro Val
Ile Pro Gln Asp Trp 355 360 365Ser Thr Glu Cys Met Thr Thr Asn Ser His Gln Val
Leu Lys Val Ile 370 375 380Phe Ala Val Ala Phe Cys Leu Ile Ser Ala Val Leu Met
Val Leu Leu385 390 395 400Phe Ile His Ile Arg Arg Gly Leu Cys Trp Gln Arg Glu
Ser Tyr Gly 405 410 415Asn Ile

### EMBODIMENTS

The following list includes particular embodiments of the present invention. The list, however, is not limiting and does not exclude the above embodiments, the above examples, or alternate embodiments, as would be appreciated by one of ordinary skill in the art.

1. A method for treating vesicle transport disorders involving SNARE complex/Snapin interaction, comprising increasing the level or restoring the activity of transmembrane prostatic acid phosphatase in a patient suffering from said disorder.

2. The method of embodiment 1, wherein the increasing the level or restoring the activity transmembrane prostatic acid phosphatase protein comprises administering a transmembrane prostatic acid phosphatase protein having a transmembrane domain and endosomal/lysosomal targeting signal in the C-terminus, or the C-terminal part thereof having a transmembrane domain and endosomal/lysosomal targeting signal in the C-terminus, to the patient suffering from said disorder.

3. The method of embodiment 2, wherein the transmembrane prostatic acid phosphatase protein or the C-terminal part thereof is administered in a liposome.

4. The method of embodiment 2, wherein the transmembrane prostatic acid phosphatase protein or the C-terminal part thereof is administered in an exosome.

5. The method of embodiment 2, wherein the transmembrane prostatic acid phosphatase protein or the C-terminal part thereof is administered as coupled to an antibody specific to a cancer cell.

6. The method of embodiment 1, wherein the increasing the level or restoring the activity transmembrane prostatic acid phosphatase protein comprises giving to the patient suffering from said disorder gene therapy by administering a gene encoding the transmembrane prostatic acid phosphatase protein or the C-terminal part thereof.

7. The method of embodiment 6, wherein the transmembrane prostatic acid phosphatase protein or the C-terminal part thereof is administered by administering a nucleic acid comprising the coding sequence thereof to a carcinoma and allowing the coding sequence to be expressed in the carcinoma.

8. The method of embodiment 1, wherein the increasing the level or restoring the activity transmembrane prostatic acid phosphatase protein comprises administering to the patient suffering from said disorder an agent capable of inducing the expression of the transmembrane prostatic acid phosphatase protein.

9. The method of any one or more of embodiments 1-8, wherein the vesicle transport disorder is a metabolic disorder related to glucose or lipid metabolism.

10. The method of any one or more of embodiments 1-9, wherein the vesicle transport disorder is a disorder related to cancer.

11. The method of embodiment 10, wherein the disorder related to cancer is selected from carcinoma, bladder cancer, pancreatic cancer, prostate cancer, and leukemia.

12. The method of any one or more of embodiments 1-8, wherein the vesicle transport disorder is selected from overactivity of antigen presentation, immune response, inflammation response and autoimmune response, overactivity of ion channels in prostate, kidneys and lungs; prostatitis; myopathy; inflammatory, immunodefence and autoimmune diseases.

13. The method of any one or more of embodiments 1-8, wherein the vesicle transport disorder is a bone marrow proliferation disorder.

14. The method of any one or more of embodiments 1-8, wherein the vesicle transport disorder is disturbed neurotransmission.

15. The method of embodiment 14, wherein the vesicle transport disorder is schizophrenia.

16. The method of any one or more of embodiments 1-8, wherein the vesicle transport disorder is neurodegeneration.

17. The method of any one or more of embodiments 1-16, wherein the transmembrane prostatic acid phosphatase is a human transmembrane prostatic acid phosphatase.

18. The method of any one or more of embodiments 1-17, wherein the region containing the transmembrane domain and the endosomal/lysosomal targeting signal in the C-terminus has at least 80% homology at amino acid level to the amino acid sequence of amino acids 383-418 of SEQ NO: 1.

19. The method of embodiment 18, wherein the region containing the transmembrane domain and the endosomal/lysosomal targeting signal in the C-terminus has the amino acid sequence of amino acids 383-418 of SEQ NO: 1.

20. The method of any one or more of embodiments 1-19, wherein the transmembrane prostatic acid phosphatase protein has at least 80% homology at amino acid level to the amino acid sequence of SEQ ID NO: 1.

21. The method of embodiment 20, wherein the transmembrane prostatic acid phosphatase protein has the amino acid sequence of SEQ ID NO: 1.

What is claimed is:

1. A method for treating vesicle transport disorders involving SNARE complex/Snapin interaction, comprising increasing the level or restoring the activity of transmembrane prostatic acid phosphatase in a patient suffering from said disorder.

2. The method of claim 1, wherein the increasing the level or restoring the activity transmembrane prostatic acid phosphatase protein comprises administering a transmembrane prostatic acid phosphatase protein having a transmembrane domain and endosomal/lysosomal targeting signal in the C-terminus, or the C-terminal part thereof having a transmembrane domain and endosomal/lysosomal targeting signal in the C-terminus, to the patient suffering from said disorder.

3. The method of claim 2, wherein the transmembrane prostatic acid phosphatase protein or the C-terminal part thereof is administered in a liposome.

4. The method of claim 2, wherein the transmembrane prostatic acid phosphatase protein or the C-terminal part thereof is administered in an exosome.

5. The method of claim 2, wherein the transmembrane prostatic acid phosphatase protein or the C-terminal part thereof is administered as coupled to an antibody specific to a cancer cell.

6. The method of claim 1, wherein the increasing the level or restoring the activity transmembrane prostatic acid phosphatase protein comprises giving to the patient suffering from said disorder gene therapy by administering a gene encoding the transmembrane prostatic acid phosphatase protein or the C-terminal part thereof.

7. The method of claim 6, wherein the transmembrane prostatic acid phosphatase protein or the C-terminal part thereof is administered by administering a nucleic acid comprising the coding sequence thereof to a carcinoma and allowing the coding sequence to be expressed in the carcinoma.

8. The method of claim 1, wherein the increasing the level or restoring the activity transmembrane prostatic acid phosphatase protein comprises administering to the patient suffering from said disorder an agent capable of inducing the expression of the transmembrane prostatic acid phosphatase protein.

9. The method of claim 1, wherein the vesicle transport disorder is a metabolic disorder related to glucose or lipid metabolism.

10. The method of claim 1, wherein the vesicle transport disorder is a disorder related to cancer.

11. The method of claim 10, wherein the disorder related to cancer is selected from carcinoma, bladder cancer, pancreatic cancer, prostate cancer, and leukemia.

12. The method of claim 1, wherein the vesicle transport disorder is selected from overactivity of antigen presentation, immune response, inflammation response and autoimmune response, overactivity of ion channels in prostate, kidneys and lungs; prostatitis; myopathy; inflammatory, immunodefence and autoimmune diseases.

13. The method of claim 1, wherein the vesicle transport disorder is a bone marrow proliferation disorder.

14. The method of claim 1, wherein the vesicle transport disorder is disturbed neurotransmission.

15. The method of claim 14, wherein the vesicle transport disorder is schizophrenia.

16. The method of claim 1, wherein the vesicle transport disorder is neurodegeneration.

17. The method of claim 1, wherein the transmembrane prostatic acid phosphatase is a human transmembrane prostatic acid phosphatase.

18. The method of claim 1, wherein the region containing the transmembrane domain and the endosomal/lysosomal targeting signal in the C-terminus has at least 80% homology at amino acid level to the amino acid sequence of amino acids 383-418 of SEQ NO: 1.

19. The method of claim 18, wherein the region containing the transmembrane domain and the endosomal/lysosomal targeting signal in the C-terminus has the amino acid sequence of amino acids 383-418 of SEQ NO: 1.

20. The method of claim 1, wherein the transmembrane prostatic acid phosphatase protein has at least 80% homology at amino acid level to the amino acid sequence of SEQ ID NO: 1.

21. The method of claim 20, wherein the transmembrane prostatic acid phosphatase protein has the amino acid sequence of SEQ ID NO: 1.
